SEQUENCE LISTING (47 sequences)

SEQ ID NO: 1 - protein, 202 aa, Escherichia coli
MKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWL

SEQ ID NO: 2 - protein, 363 aa, Escherichia coli
MVNVKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 3 - protein, 208 aa, Escherichia coli
MMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 4 - protein, 881 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSIDSAAHHDNSTIPLDFMPRDALHGFDWSEEDDMSDGLPFLKTDPNNNGFFGDGSLLCILRSIGFKPENYTNSNVNRLPTMITDRYTLASRSTTSRLLQSYLNNFHPYCPIVHSPTLMMLYNNQIEIASKDQWQILFNCILAIGAWCIEGESTDIDVFYYQNAKSHLTSKVFESGSIILVTALHLLSRYTQWRQKTNTSYNFHSFSIRMAISLGLNRDLPSSFSDSSILEQRRRIWWSVYSWEIQLSLLYGRSIQLSQNTISFPSSVDDVQRTTTGPTIYHGIIETARLLQVFTKIYELDKTVTAEKSPICAKKCLMICNEIEEVSRQAPKFLQMDISTTALTNLLKEHPWLSFTRFELKWKQLSLIIYVLRDFFTNFTQKKSQLEQDQNDHQSYEVKRCSIMLSDAAQRTVMSVSSYMDNHNVTPYFAWNCSYYLFNAVLVPIKTLLSNSKSNAENNETAQLLQQINTVLMLLKKLATFKIQTCEKYIQVLEEVCAPFLLSQCAIPLPHISYNNSNGSAIKNIVGSATIAQYPTLPEENVNNISVKYVSPGSVGPSPVPLKSGASFSDLVKLLSNRPPSRNSPVTIPRSTPSHRSVTPFLGQQQQLQSLVPLTPSALFGGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 5 - protein, 816 aa, Neurospora crassa
MPPKRKTLNAAAEANAHADGHADGNADGHVANTAASSNNARFADLTNIDTPGLGPTTTTLLVEPARSKRQRVSRACDQCRAAREKCDGIQPACFPCVSQGRSCTYQASPKKRGVQTGYIRTLELALAWMFENVARSEDALHNLLVRDAGQGSALLVGKDSPAAERLHARWATSRVNKSITRLLSGQAAQDPSEDGQSPSEDINVQDAGAKTSDFPHAPHLTFSAPKSSTAETRTLPGPVRPPISANTLENNLQPDGTGIGKLPPNHWRLLDIYFSYTHSWLPILEKKDMYQALYQYSEQGSLLPSANVESGVHAELWSALALASFQAAATAASSATGPASAAHGHDNAINPSPADISDTARKLIPLESGPFQVQHCRALLLLCLVSLGRDDWESAWLLVGFAVRVLLVVRTQLPPDDDRPRPRMRALLVACFIVDTIVSMRHNVPAHLKPDDIADLPLPEDGQDQWEPWTPCEGLGGEHTMLQMLRNPAYPLSTFNHLYGVTKLVALELLPRIRTSSQNAPLEFRSRLQQVIGHNSPFSVFVLSQDTASAFVPTAYLTRTVYLWAAAFSEPLNEHYSHLLIETLDQYQKRFGTYAIPPLIPSLLDSLLALKKQSHSSERHRRHLEELFPAYSSIWPRGGRHSNTGLQPIRQLELPPTATATASIMPHVMEQPLSTSINPVNDRFNGIPNPTPYNSDAALDAITQTNDYGSVNTHGILSTYPPPATHLNEASVALAPGGAPPRPPPPYVDSTTNHPPYHSNLVPMANFGYSTVDYDAMVDDLASIEYTDAVDVDPQFMTNLGFVPGCNFSDISTYEQ

SEQ ID NO: 6 - protein, 93 aa, herpes simplex virus
EFPGIRRPAGIPGDLAPPTDVSLGDELHLDGEDVAMAHADALDDFDLDMLGDGDSPGPGFTPHDSAPYGALDMADFEFEQMFTDALGIDEYGG

SEQ ID NO: 7 - protein, 295 aa, synthetic construct
  REGION 1..295: note = amino acid sequence of LexA/VP16 fusion
MKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLEFPGIRRPAGIPGDLAPPTDVSLGDELHLDGEDVAMAHADALDDFDLDMLGDGDSPGPGFTPHDSAPYGALDMADFEFEQMFTDALGIDEYGG

SEQ ID NO: 8 - protein, 436 aa, synthetic construct
  REGION 1..436: note = amino acid sequence of lactose repressor/VP16 fusion
MVNVKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRAPPTDVSLGDELHLDGEDVAMAHADALDDFDLDMLGDGDSPGPGFTPHDSAPYGALDMADFEFEQMFTDALGIDEYGG

SEQ ID NO: 9 - protein, 336 aa, synthetic construct
  REGION 1..336: note = amino acid sequence of tetracycline-controlled transactivator (tTA)
MMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 10 - protein, 232 aa, synthetic construct
  REGION 1..232: note = amino acid sequence of GAL4/VP16 fusion protein
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFPGIWAPPTDVSLGDELHLDGEDVAMAHADALDDFDLDMLGDGDSPGPGFTPHDSAPYGALDMADFEFEQMFTDALGIDEYGG

SEQ ID NO: 11 - protein, 382 aa, synthetic construct
  REGION 1..382: note = amino acid sequence of LexA/QF fusion protein
MPPKKKRKVEDPMKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWLGRQLELPPTATATASIMPHVMEQPLSTSINPVNDRFNGIPNPTPYNSDAALDAITQTNDYGSVNTHGILSTYPPPATHLNEASVALAPGGAPPRPPPPYVDSTTNHPPYHSNLVPMANFGYSTVDYDAMVDDLASIEYTDAVDVDPQFMTNLGFVPGCNFSDINTYEQ

SEQ ID NO: 12 - skipped

SEQ ID NO: 13 - protein, 500 aa, Homo sapiens
MATAASNPYLPGNSLLAAGSIVHSDAAGAGGGGGGGGGGGGGGAGGGGGGMQPGSAAVTSGAYRGDPSSVKMVQSDFMQGAMAASNGGHMLSHAHQWVTALPHAAAAAAAAAAAAVEASSPWSGSAVGMAGSPQQPPQPPPPPPQGPDVKGGAGRDDLHAGTALHHRGPPHLGPPPPPPHQGHPGGWGAAAAAAAAAAAAAAAAHLPSMAGGQQPPPQSLLYSQPGGFTVNGMLSAPPGPGGGGGGAGGGAQSLVHPGLVRGDTPELAEHHHHHHHHAHPHPPHPHHAQGPPHHGGGGGGAGPGLNSHDPHSDEDTPTSDDLEQFAKQFKQRRIKLGFTQADVGLALGTLYGNVFSQTTICRFEALQLSFKNMCKLKPLLNKWLEEADSSTGSPTSIDKIAAQGRKRKKRTSIEVSVKGALESHFLKCPKPSAQEITNLADSLQLEKEVVRVWFCNRRQKEKRMTPPGIQQQTPDDVYSQVGTVSADTPPPHHGLQTSVQ

SEQ ID NO: 14 - protein, 443 aa, Homo sapiens
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 15 - DNA, 15 bp, synthetic construct
  misc_feature 1..15: note = DNA binding site of NFIC
  misc_feature 6..10: note = n is a, c, g, or t
  source 1..15: mol_type = other DNA
ttggcnnnnngccaa

SEQ ID NO: 16 - protein, 508 aa, Homo sapiens
MYSSPLCLTQDEFHPFIEALLPHVRAFAYTWFNLQARKRKYFKKHEKRMSKDEERAVKDELLGEKPEVKQKWASRLLAKLRKDIRPECREDFVLSITGKKAPGCVLSNPDQKGKMRRIDCLRQADKVWRLDLVMVILFKGIPLESTDGERLVKAAQCGHPVLCVQPHHIGVAVKELDLYLAYFVRERDAEQSGSPRTGMGSDQEDSKPITLDTTDFQESFVTSGVFSVTELIQVSRTPVVTGTGPNFSLGELQGHLAYDLNPASTGLRRTLPSTSSSGSKRHKSGSMEEDVDTSPGGDYYTSPSSPTSSSRNWTEDMEGGISSPVKKTEMDKSPFNSPSPQDSPRLSSFTQHHRPVIAVHSGIARSPHPSSALHFPTTSILPQTASTYFPHTAIRYPPHLNPQDPLKDLVSLACDPASQQPGPLNGSGQLKMPSHCLSAQMLAPPPPGLPRLALPPATKPATTSEGGATSPTSPSYSPPDTSPANRSFVGLGPRDPAGIYQAQSWYLG

SEQ ID NO: 17 - DNA, 15 bp, synthetic construct
  misc_feature 1..15: note = DNA binding site of NFIC
  misc_feature 6..10: note = n is a, c, g, or t
  source 1..15: mol_type = other DNA
ttggcnnnnngccaa

SEQ ID NO: 18 - protein, 502 aa, Homo sapiens
MYSPYCLTQDEFHPFIEALLPHVRAFSYTWFNLQARKRKYFKKHEKRMSKDEERAVKDELLGEKPEIKQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGVTIKELDLYLAYFVHTPESGQSDSSNQQGDADIKPLPNGHLSFQDCFVTSGVWNVTELVRVSQTPVATASGPNFSLADLESPSYYNINQVTLGRRSITSPPSTSTTKRPKSIDDSEMESPVDDVFYPGTGRSPAAGSSQSSGWPNDVDAGPASLKKSGKLDFCSALSSQGSSPRMAFTHHPLPVLAGVRPGSPRATASALHFPSTSIIQQSSPYFTHPTIRYHHHHGQDSLKEFVQFVCSDGSGQATGQPNGSGQGKVPGSFLLPPPPPVARPVPLPMPDSKSTSTAPDGAALTPPSPSFATTGASSANRFVSIGPRDGNFLNIPQQSQSWFL

SEQ ID NO: 19 - skipped

SEQ ID NO: 20 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 21 - DNA, 10 bp, synthetic construct
  misc_feature 1..10: note = DNA binding site of MEF2A
  source 1..10: mol_type = other DNA
ytawwwwtar

SEQ ID NO: 22 - protein, 506 aa, Homo sapiens
MGRKKIQITRIMDERNRQVTFTKRKFGLMKKAYELSVLCDCEIALIIFNSSNKLFQYASTDMDKVLLKYTEYNEPHESRTNSDIVEALNKKEHRGCDSPDPDTSYVLTPHTEEKYKKINEEFDNMMRNHKIAPGLPPQNFSMSVTVPVTSPNALSYTNPGSSLVSPSLAASSTLTDSSMLSPPQTTLHRNVSPGAPQRPPSTGNAGGMLSTTDLTVPNGAGSSPVGNGFVNSRASPNLIGATGANSLGKVMPTKSPPPPGGGNLGMNSRKPDLRVVIPPSSKGMMPPLSEEEELELNTQRISSSQATQPLATPVVSVTTPSLPPQGLVYSAMPTAYNTDYSLTSADLSALQGFNSPGMLSLGQVSAWQQHHLGQAALSSLVAGGQLSQGSNLSINTNQNISIKSEPISPPRDRMTPSGFQQQQQQQQQQQPPPPPQPQPQPPQPQPRQEMGRSPVDSLSSSSSSYDGSDREDPRGDFHSPIVLGRPPNTEDRESPSVKRMRMDAWV

SEQ ID NO: 23 - skipped

SEQ ID NO: 24 - protein, 345 aa, Homo sapiens
MLMFDPVPVKQEAMDPVSVSYPSNYMESMKPNKYGVIYSTPLPEKFFQTPEGLSHGIQMEPVDLTVNKRSSPPSAGNSPSSLKFPSSHRRASPGLSMPSSSPPIKKYSPPSPGVQPFGVPLSMPPVMAAALSRHGIRSPGILPVIQPVVVQPVPFMYTSHLQQPLMVSLSEEMENSSSSMQVPVIESYEKPISQKKIKIEPGIEPQRTDYYPEEMSPPLMNSVSPPQALLQENHPSVIVQPGKRPLPVESPDTQRKRRIHRCDYDGCNKVYTKSSHLKAHRRTHTGEKPYKCTWEGCTWKFARSDELTRHFRKHTGIKPFQCPDCDRSFSRSDHLALHRKRHMLV

SEQ ID NO: 25 - protein, 1437 aa, synthetic construct
  REGION 1..1437: note = amino acid sequence of dCas9/VP64
MKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVDALDDFDLDMLGSDALDDFDLDMLGSDALDDFDLDMLGSDALDDFDLDMLYID

SEQ ID NO: 26 - DNA, 103 bp, synthetic construct
  misc_feature 1..103: note = 5-repeat GAL4 upstream activating sequence
  source 1..103: mol_type = other DNA
cggagtactgtcctccgagcggagtactgtcctccgactcgagcggagtactgtcctccgatcggagtactgtcctccgcgaattccggagtactgtcctccg

SEQ ID NO: 27 - DNA, 271 bp, synthetic construct
  misc_feature 1..271: note = Tet response element (TRE)
  source 1..271: mol_type = other DNA
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagaga

SEQ ID NO: 28 - DNA, 78 bp, synthetic construct
  misc_feature 1..78: note = LacI promoter sequence
  source 1..78: mol_type = other DNA
gacaccatcgaatggcgcaaaacctttcgcggtatggcatgatagcgcccggaagagagtcaattcagggtggtgaat

SEQ ID NO: 29 - DNA, 257 bp, Mus musculus
  source 1..257: mol_type = genomic DNA
ggtctgaaaaggatttggagaaggggagctgaattcatttgcttttgtctgttaccagctctgggggcagagagagagccatcccctgggaacagcctgagaattcccacttcccctgaggagccctcccttcttaggccctccagatggtagtgtggacaaaaggcaataattagcatgagaatcggcctccctcccagaggatgaggtcatcggccttggccttgggtggggaggcggagactgatctgaggagt

SEQ ID NO: 30 - DNA, 507 bp, synthetic construct
  misc_feature 1..507: note = Glial Fibrillary Acidic Protein (GFAP) promoter sequence
  source 1..507: mol_type = other DNA
acatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagagtcgacggatccggggaattccccagtctcaggatccaccatgggg

SEQ ID NO: 31 - DNA, 151 bp, Mus musculus
  source 1..151: mol_type = genomic DNA
atccactggaaacgtcttgatgtgcagcaacagcttagaggggggctcaggtttctgtggcgttggctatatttatctctgggttcatgccagcagggagggtttaaatggcacccagcagttggtgtgaggggctgcgggagcttggggg

SEQ ID NO: 32 - DNA, 142 bp, Mus musculus
  source 1..142: mol_type = genomic DNA
ccactacgggtctaggctgcccatgtaaggaggcaaggcctggggacacccgagatgcctggttataattaacccagacatgtggctgcccccccccccccaacacctgctgcctctaaaaataaccctgtccctggtggat

SEQ ID NO: 33 - DNA, 771 bp, synthetic construct
  misc_feature 1..771: note = Murine muscle creatine kinase/alpha-myosin heavy chain (MHCK7) promoter
  source 1..771: mol_type = other DNA
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 34 - protein, 16 aa, synthetic construct
  REGION 1..16: note = QBEnd10-binding epitope
ELPTQGTFSNVSTNVS

SEQ ID NO: 35 - protein, 214 aa, synthetic construct
  REGION 1..214: note = SHP-1 SH2 complete domain
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYY

SEQ ID NO: 36 - protein, 97 aa, synthetic construct
  REGION 1..97: note = SHP-1 SH2 1 sequence
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 37 - protein, 104 aa, synthetic construct
  REGION 1..104: note = SHP-1 SH2 2 sequence
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 38 - protein, 97 aa, synthetic construct
  REGION 1..97: note = SHP-2 first SH2 domain
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 39 - protein, 105 aa, synthetic construct
  REGION 1..105: note = SHP-2 second SH2 domain
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 40 - protein, 211 aa, synthetic construct
  REGION 1..211: note = SHP-2 both SH2 domains
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 41 - protein, 196 aa, synthetic construct
  REGION 1..196: note = truncated TGF beta receptor 2 sequence
MGWSCIILFLVATATGVHSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 42 - protein, 517 aa, synthetic construct
  REGION 1..517: note = transgene FRB-FKBP12-L3-dCasp9
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKLEYSGGGSLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGGSGGGGSGGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 43 - protein, 136 aa, synthetic construct
  REGION 1..136: note = sort-suicide gene RQR8
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 44 - protein, 20 aa, Foot-and-mouth disease virus
RAEGRGSLLTCGDVEENPGP

SEQ ID NO: 45 - protein, 20 aa, Foot-and-mouth disease virus
QCTNYALLKLAGDVESNPGP

SEQ ID NO: 46 - skipped

SEQ ID NO: 47 - DNA, 10 bp, synthetic construct
  misc_feature 1..10: note = Transcription factor C/EBP recognition sequence
  source 1..10: mol_type = other DNA
attgcgcaat

(19) United States
(12) Patent Application Publication
(10) Pub. No.: US 2024/0309400 A1
(43) Pub. Date: Sep. 19, 2024

(54) VECTORS

(71) Applicant: AUTOLUS LIMITED, London (GB)

(72) Inventors: Martin Pulé, London (GB); James Sillibourne, London (GB); Shaun Cordoba, London (GB)

(21) Appl. No.: 18/504,554

(22) Filed: Nov. 8, 2023

Related U.S. Application Data
(63) Continuation of application No. 16/759,222, filed on Apr. 24, 2020, now Pat. No. 11,851,672.

(30) Foreign Application Priority Data
Oct. 25, 2017 (GB) 1717524.1

(51) Int. Cl. C12N 15/86; A61K 35/17; A61K 48/00

(52) U.S. Cl. CPC C12N 15/86; A61K 35/17; A61K 48/00; C12N 2740/10043; C12N 2800/40

(57) ABSTRACT

The present invention provides a kit of vectors for transducing an immune cell with multiple transgenes comprising: (i) a first vector which comprises a first transgene and a nucleotide sequence encoding a transcription factor and; and (ii) a second vector which comprises a second transgene wherein expression of the second transgene within a host cell is dependent upon expression of the transcription factor.

### CROSS-REFERENCE TO RELATED APPLICATIONS

This is a Continuation of U.S. application Ser. No. 16/759,222, filed Apr. 24, 2020, which is a U.S. National Phase of International Application No. PCT/GB2018/053088, filed Oct. 24, 2018, which claims priority to Great Britain Application No. 1717524.1, filed Oct. 25, 2017.

### INCORPORATION BY REFERENCE OF THE SEQUENCE LISTING

This application contains, as a separate part of disclosure, a Sequence Listing in computer-readable form (Filename: 53693A_SeqListing.xml; 62,157 bytes—XML file dated Nov. 1, 2023) which is incorporated by reference herein in its entirety.

### FIELD OF THE INVENTION

The present invention relates to the transduction or transfection of cells with multiple vectors. In particular, the present invention relates to kit of vectors in which a first vector expresses a transcription factor upon which the expression of a transgene from a second vector is dependent. Cascading or rotating transcriptional circuits are also described, based on this principle.

### BACKGROUND TO THE INVENTION

The genetic modification of therapeutic immune cells often involves the introduction of genes which re-target T-cells to, for example, cancer antigens. Such re-targeting genes include chimeric antigen receptors (CARs) and transgenic T-cell receptors (TCRs). To increase the potency and safety of engineered T-cells, such therapeutic approaches are increasing in complexity with introduction of multiple additional genetic elements.

For example, the cells may also be modified to introduce genes with other functions, such as genes which enhance proliferation, survival or allow pharmacological control of immune cells. Alternatively, or in addition, the cells may be engineered to express marker genes. These typically encode surface expression proteins which allow selection or transduced cells during cell production. Alternatively, or in addition, the cells may be engineered to express antibiotic resistance genes which allow selection during production by exposure to an antibiotic.

There is therefore a need to introduce multiple transgenes into therapeutic immune cells. Gene vectors which are used to introduce transgenes into therapeutic immune cells are typically integrating vectors since expression must be long-lived. To date, the vector system of choice is retroviral vectors, which include gamma-retroviral vectors and lentiviral vectors. Vector systems can allow expression of multiple genes from a single cassette using internal ribosome entry sequences or self-cleaving protein motifs such as FMD-2A like peptides. However, expression cassettes encoding multiple genes often cannot be expressed by a single vector because they are too large: the packaging limit of retroviral vectors varies with subtype and pseudotype but is typically under 10 kb.

One solution to this limitation is to simply transduce a T-cell with multiple vectors. However, as transduction is not 100 percent efficient, this creates a mixed population of cells with multiple combinations of vectors. Only a subset of the cells will have been successfully transduced with all vectors.

There is therefore a need for alternative vector systems capable of expressing multiple transgenes.

### DESCRIPTION OF THE FIGURES

FIG. 1—A schematic diagram illustrating kit of vectors with a marked primary cassette and a dependent secondary cassette

In this scenario, a primary cassette is driven by a constitutively active promoter and encodes a primary transgenic element (transgenes X and Y separated by a 2A peptide) and a transcription factor (T). A secondary cassette is driven by a promoter sequence (pT) dependent on the transcription factor supplied by the primary cassette. The secondary cassette encodes ancillary transgenes A and B. The primary cassette also encodes a marker gene (M), so that cells which do not contain the primary cassette, and therefore do not express transgenes, can be removed.

FIG. 2—A schematic diagram illustrating kit of vectors with a primary cassette and a dependent marked secondary cassette

In this scenario, a primary cassette is driven by a constitutively active promoter and encodes a primary transgenic element (transgenes X and Y separated by a 2A peptide) and a transcription factor (T). A secondary cassette is driven by a promoter sequence (pT) dependent on the transcription factor supplied by the primary cassette. The secondary cassette encodes ancillary transgenes A and B. The secondary cassette also encodes a marker gene, so that sorting on the marker gene results in the selection of cells expressing both the primary and secondary cassettes only.

FIG. 3—A schematic diagram illustrating kit of vectors with a primary cassette and multiple dependent cassettes

In this scenario, a primary cassette is driven by a constitutively active promoter and encodes a primary transgenic element (transgenes X and Y separated by a 2A peptide) and a transcription factor (T). A secondary cassette is driven by a promoter sequence (pT) dependent on the transcription factor supplied by the primary cassette. The secondary cassette encodes ancillary transgenes A and B. A tertiary cassette is also driven by a promoter sequence (pT) dependent on the transcription factor and encodes ancillary transgenes P and Q. The primary cassette also encodes a marker gene (M), so that cells which do not contain the primary cassette, and therefore do not express transgenes, can be removed.

FIG. 4—A schematic diagram illustrating kit of vectors with a primary cassette and iterative dependent cassettes

In this scenario, a primary cassette is driven by a constitutively active promoter and encodes a primary transgenic element (transgenes X and Y separated by a 2A peptide) and a transcription factor (T). A secondary cassette is driven by a promoter sequence (pT1) dependent on the transcription factor supplied by the primary cassette. The secondary cassette encodes ancillary transgenes A and B. A tertiary cassette is driven by a promoter sequence (pT2) dependent on the transcription factor supplied by the secondary cassette. The secondary cassette encodes ancillary transgenes P and Q. In this scenario, an iterative dependence of expression is required down the chains of expression cassettes. If a marker is expressed by the last expression cassette, sorting will select cells which express all cassettes.

FIG. 5—A schematic diagram illustrating kit of vectors with circular dependent cassettes

In this scenario, a primary cassette is driven by a promoter sequence (pT2) dependent on a transcription factor supplied by the secondary cassette. The primary cassette encodes transgenes X and Y separated by a 2A peptide and a first transcription factor (T1). A secondary cassette is driven by a promoter sequence (pT1) dependent on the transcription factor supplied by the primary cassette. The secondary cassette encodes ancillary transgenes A and B and a second transcription factor (T2). In this case if cells are transduced with only one of the two cassettes, expression is silent. If a cell is transduced with both cassettes, a small leak of expression present in all such systems would be amplified such that both cassettes are expressed.

FIG. 6—Diagram illustrating the structure of the GAL4 system constructs described in Example 2

The GAL4 transcription factor sequences were downstream of RQR8 in the retroviral vector SFGmR, with a 2a self-cleaving peptide sequence enabling the expression of both proteins (top image). The GAL4 UAS sequence was cloned into the SIN retroviral construct pSuper, placing expression of the V5-tagged anti-CD19 second generation CAR under the control of the inducible promoter. An internal PGK promoter enabled the expression HA8 a cell surface marker used to detect transduced cells (bottom image).

FIG. 7—Quantification of RQR8 and V5 double positive PBMCs

Quantification of the percentage of RQR8 and V5 double positive cells indicated that expression required the presence of active VP16-GAL4 transcription factor with the GAL4 UAS promoter (Example 2).

FIG. 8—Quantification of the V5-tagged CAR MFI

Quantification of the median fluorescent intensity of the V5-tagged CAR showed that there was a 15-fold increase in expression of the CAR when cells were co-transduced with the GAL4 UAS promoter and VP16-GAL4 transcription factor compared to co-transduction with the GAL4 DBD or the promoter alone. Statistical analysis: 1way ANOVA Dunnett's multiple comparisons test*p<0.05 (Example 2).

FIG. 9—Structure of LexA constructs described in Example 3

The top image illustrates the retroviral construct containing the sort selection marker RQR8 and the chimeric VP16-LexA transcription factor. The lower cartoon shows the structure of the SIN retroviral construct with the LexA UAS driving the expression of the V5-tagged anti-CD19 second generation CAR and the internal human PGK promoter controlling the expression of an HA8 marker (HA epitope presented on a CD8a stalk).

FIG. 10—Quantification of MFI of V5-tagged CAR.

Quantification of the MFI of the V5-tagged antib-CD19 CAR demonstrated that expression increased when PBMCs were co-transduced with the LexA UAS promoter construct and the active VP16-LexA transcription factor. Expression of the V5-tagged anti-CD19 CAR increased in PBMCs co-transduced with the LexA UAS promoter and the active VP16-LexA transcription factor, but not with the inactive LexA DNA-binding domain alone transcription factor.

FIG. 11—Diagram of QF2 system described in Example 4

Similar to the GAL4 and LexA systems, the QF2 system utilises a retroviral construct to express RQR8 sort selection marker and the QF2 transcription factor and a SIN retroviral construct to control the expression of a V5-tagged CAR from the QUAS promoter. Expression of a HA8 marker in the SIN retroviral construct is driven by an internal PGK promoter.

FIG. 12—Quantification of V5-tagged anti-CD19 CAR MFI

Quantification of the median fluorescence intensity of the V5-tagged anti-CD19 CAR showed that the expression was 8-fold higher in PBMCs co-transduced with the active QF2 transcription factor and QUAS promoter construct, indicating that the QF2 transcription factor is functional in PBMCs and able to initiate transcription from its cognate UAS.

FIG. 13—Diagram of circular transcriptional dependent circuit constructs described in Example 5

A pair of SIN retroviral constructs with opposing UAS and artificial transcription factors are used to set up a self-amplifying circuit that is initiated by leaky expression from the SIN retroviral constructs. The top construct contains a LexA UAS that drives expression of the sort selection marker RQR8 and the VP16-GAL4 transcription factor, while to bottom construct has a GAL4 UAS controlling expression of a V5-tagged anti-CD19 CAR, HA8 marker and LexA transcription factor. When a cell is transduced with both constructs, leaky transcription should initiate expression of all components and set up a self-amplifying circular transcriptional circuit.

### SUMMARY OF ASPECTS OF THE INVENTION

The present inventors have developed a vector system comprising two or more vectors in which expression of a transgene from one vector is dependent on expression of a transcription factor from another vector. Successful transduction with both vectors can be deduced via expression of a marker gene. It is therefore possible to detect and sort for cells expressing both or all of the vectors in the kit.

Thus in a first aspect, the present invention provides a kit of vectors for transducing an immune cell with multiple transgenes comprising:
(i) a first vector which comprises a first transgene and a nucleotide sequence encoding a transcription factor and; and
(ii) a second vector which comprises a second transgene
wherein expression of the second transgene within a host cell is dependent upon expression of the transcription factor.

The second vector may also comprise a marker gene, the expression of which is dependent on the expression of the transcription factor. Expression of the marker by a cell transduced with the kit of vector indicates that the cell has been successfully transduced with both the first and second vector.

The kit of vectors may comprise more than one dependent vector. For example, the kit of vectors may comprise:
(i) a first vector which comprises a first transgene and a nucleotide sequence encoding a transcription factor; and
(ii) a second vector which comprises a second transgene; and
(iii) a third vector which comprises a third transgene
wherein expression of the second and third transgenes within a host cell is dependent upon expression of the transcription factor.

The kit of vectors may comprise iterative dependent vectors. For example, the kit of vectors may comprise:
(i) a first vector which comprises a first transgene and a nucleotide sequence encoding a first transcription factor; and
(ii) a second vector which comprises a second transgene and a nucleotide sequence encoding a second transcription factor; and
(iii) a third vector which comprises a third transgene
wherein expression of the second transgene within a host cell is dependent upon expression of the first transcription factor, and expression of the third transgene within a host cell is dependent upon expression of the second transcription factor.

In this embodiment, the third vector may also comprise a marker gene, the expression of which is dependent on the expression of the second transcription factor. Expression of the marker by a cell transduced with the kit of vector indicates that the cell has been successfully transduced with all three vectors.

The kit of vectors may comprise inter-dependent vectors. For example, the kit of vectors may comprise:
(i) a first vector which comprises a first transgene and a nucleotide sequence encoding a first transcription factor; and
(ii) a second vector which comprises a second transgene and a nucleotide sequence encoding a second transcription factor;
wherein expression of the second transgene within a host cell is dependent upon expression of the first transcription factor, and expression of the first transgene within a host cell is dependent upon expression of the second transcription factor.

One or more of the vector(s) in the kit may also comprises a marker gene.

The vectors may be plasmids, transposons, retroviral vectors or lentiviral vectors.

The transcription factor may be a prokaryotic or eukaryotic transcription factor that is functional in human cells. Examples of such transcription factors include LexA, TetR and LacI from bacteria; GAL4 from yeast; or QF, a transcriptional activator binding to QF Upstream Activating Sequences (QUAS) present in the qa cluster from Neurospora.

The transcription factor may be an artificial factor consisting of a DNA-binding domain, recognising a unique DNA sequence present in a promoter, and a transactivation domain derived from another transcription factor. Examples of such transcription factors include fusions between the DNA-binding domain of GAL4, LexA, Lac repressor, or QF and the transactivation domain of herpes simplex virus virion protein 16 (VP16).

The transcription factor may be a tissue-specific transcription factor that is not expressed in an immune cell and could be used to drive transcription from its cognate tissue-specific promoter. Examples of tissue-specific transcription factors include those from muscle cells such as MyoD, muscle enhancer factor 2 (MEF2) and Krueppel-like factor 3 (KLF3) or those from neuronal cells such as nuclear factor 1C (NF1C) and nuclear factor 1X (NF1X) from astrocytes or Brain-1 (Brn-1) and Brain-2 (Brn-2) from glial cells.

The transcription factor may be an artificial transcription factor generated by creating a fusion between: a zinc finger DNA-binding domain; or a transcription factor-like effector (TALE) DNA-binding domain; or a catalytically inactive form of the RNA-guided endonuclease Cas9 fused to the transactivation domain of VP16.

The first vector may comprise a constitutively active promoter.

One or more of the transgene(s) may encode a chimeric antigen receptor (CAR) or a T-cell receptor (TCR).

In a second aspect, the present invention provides a cell. The cell may comprise a transgene and a nucleotide sequence encoding a non-endogenous transcription factor, wherein expression of the transgene is dependent upon expression of the non-endogenous transcription factor.

The cell may be transfected or transduced with a kit of vectors according to the first aspect of the invention.

The cell may be an immune cell, such as a T-cell or natural killer (NK) cell.

In a third aspect, the present invention provides a method for making a cell according to the second aspect of the invention which comprises the step of transducing or transfecting a cell with a kit of vectors according to the first aspect of the invention.

In a fourth aspect there is provided a method for making a cell composition expressing a kit of vectors according to the first aspect of the invention, which comprises the following steps:
(i) transducing or transfecting a cell with a kit of vectors according to the first aspect of the invention wherein one of the vectors comprises a marker gene; and
(ii) selecting cells which express the marker gene
wherein expression of the marker gene by a cell indicates that the comprises all of the vectors in the kit.

The cell or cells may be transduced or transfected ex vivo.

In a fifth aspect, the present invention provides a pharmaceutical composition comprising a plurality of cells according to the second aspect of the invention.

In a sixth aspect, the present invention provides a pharmaceutical composition according to the fifth aspect of the invention for use in treating and/or preventing a disease.

In a seventh aspect, the present invention provides a method for treating and/or preventing a disease, which comprises the step of administering a pharmaceutical composition according to the fifth aspect of the invention to a subject.

The method may comprise the following steps:
(i) isolation of a cell containing sample from a subject;
(ii) transduction or transfection of the cells with a kit of vectors according to the first aspect of the invention; and
(iii) administering the cells from step (ii) to the subject.

In an eighth aspect, the present invention provides the use of a pharmaceutical composition according to the fifth aspect of the invention in the manufacture of a medicament for the treatment and/or prevention of a disease.

The disease may be a cancer.

The present invention therefore provide a kit of vectors enabling flexible, modular expression of multiple transgenes in a cell. If one cassette is dependent on a transcription factor for expression and the other cassette supplies the transcription factor, a transcriptional circuit can be established whereby expression of one cassette is dependent on expression of the other cassette. It is therefore possible to detect and select for cells have been successfully transduced with both or all vectors in the kit and therefore express all of the transgenes in the kit.

In addition to addressing the issue of packaging limits for single retroviral vectors, the system of the invention also offers other advantages. A modular approach is intrinsically desirable as it enables different mixtures of transgenes to be selected according for example to a patient's disease, morbidity etc. The system of the invention also facilitates the generation of a therapeutic product whereby a proportion of the immune cells express transgenes from one vector, and a proportion of immune cells express both.

### DETAILED DESCRIPTION

### Vectors

The present invention relates to a kit of vectors. A vector is used to introduce one or more nucleic acid sequence(s) into a host cell so that it expresses the protein(s) encoded by the nucleic acid sequence(s).

The vector may, for example, be a plasmid or a viral vector, such as a retroviral vector or a lentiviral vector, or a transposon based vector or synthetic mRNA.

The vector may be capable of transfecting or transducing an immune cell such as a T-cell or a natural killer (NK) cell.

### Transcription Factor

At least one vector in the kit of the invention comprises a nucleic acid encoding a transcription factor.

A transcription factor is a protein which controls the rate of transcription of genetic information from DNA to messenger RNA, by binding to a specific DNA sequence and regulate the expression of a gene which comprises or is adjacent to that sequence.

Transcription factors work by promoting (as an activator), or blocking (as a repressor) the recruitment of RNA polymerase.

Transcription factors contain at least one DNA-binding domain (DBD), which attaches to either an enhancer or promoter region of DNA. Depending on the transcription factor, the transcription of the adjacent gene is either up-or down-regulated. Transcription factors also contain a trans-activating domain (TAD), which has binding sites for other proteins such as transcription co-regulators.

Transcription factors use a variety of mechanisms for the regulation of gene expression, including stabilizing or blocking the binding of RNA polymerase to DNA, or catalyzing the acetylation or deacetylation of histone proteins. The transcription factor may have histone acetyltransferase (HAT) activity, which acetylates histone proteins, weakening the association of DNA with histones and making the DNA more accessible to transcription, thereby up-regulating transcription. Alternatively the transcription factor may have histone deacetylase (HDAC) activity, which deacetylates histone proteins, strengthening the association of DNA with histones and making the DNA less accessible to transcription, thereby down-regulating transcription. Another mechanism by which they may function is by recruiting coactivator or corepressor proteins to the transcription factor DNA complex.

Some examples of specific transcription factors are given in the table below:

| | Structural | Recognition | Binds |
|---|---|---|---|
| Factor | type | sequence | as |
| SP1 | Zinc finger | 5′-GGGCGG-3′ | Monomer |
| AP-1 | Basic | 5′-TGA(G/C)TCA-3′ | Dimer |
| | zipper | | |
| C/EBP | Basic | 5′-ATTGCGCAAT-3′ | Dimer |
| | zipper | (SEQ ID No. 47) | |
| Heat | Basic | 5′-XGAAX-3′ | Trimer |
| shock | zipper | | |
| factor | | | |
| ATF/ | Basic | 5′-TGACGTCA-3′ | Dimer |
| CREB | zipper | | |
| c-Myc | Basic | 5′-CACGTG-3′ | Dimer |
| | helix- | | |
| | loop-helix | | |
| Oct-1 | Helix- | 5′-ATGCAAAT-3′ | Monomer |
| | turn-helix | | |
| NF-1 | Novel | 5′-TTGGCXXXXXGCCAA-3′ | Dimer |

The transcription factor of the present invention may be constitutively active or conditionally active, i.e. requiring activation.

In the kit of vectors of the present invention, a constitutively active transcription factor modulates expression of the second transgene as soon as it is produced, without requiring an additional step such as cleavage from a membrane-tethered position in the cell or dimerization induced by addition of a dimerising agent.

### Transcription Factors Applied to Human Immune Cells

DNA binding elements from some prokaryotic transcription factors can function as part of a transcriptional system in human immune cells. Those with demonstrated functional activity in human cells include GAL4 (from the galactose operon), LexA (the lex operon), LacI (the lactose operon repressor), TetR (the tetracycline sensitive repressor) and the Q system (which controls 5 quinic acid catabolism

The amino acid sequence of LexA from Escherichia coli is available from Uniprot (accession number Q1R3P3) and is shown below as SEQ ID No 1.

Amino acid sequence of LexA repressor
SEQ ID No. 1
MKALTARQQEVFDLIRDHISQTGMPPTRAEIAQRLGFRSPNAAEEHLKA
LARKGVIEIVSGASRGIRLLQEEEEGLPLVGRVAAGEPLLAQQHIEGHY
QVDPSLFKPNADFLLRVSGMSMKDIGIMDGDLLAVHKTQDVRNGQVVVA
RIDDEVTVKRLKKQGNKVELLPENSEFKPIVVDLRQQSFTIEGLAVGVI
RNGDWL

The amino acid sequence of the lactose operon repressor is available from Uniprot (Accession number F4VAC4) and is shown below as SEQ ID No. 2.

Amino acid sequence of Lactose operon repressor
SEQ ID No. 2
MVNVKPVTLYDVAEYAGVSYQTVSRVVNQASHVSAKTREKVEAAMAELN
YIPNRVAQQLAGKQSLLIGVATSSLALHAPSQIVAAIKSRADQLGASVV
VSMVERSGVEACKAAVHNLLAQRVSGLIINYPLDDQDAIAVEAACTNVP
ALFLDVSDQTPINSIIFSHEDGTRLGVEHLVALGHQQIALLAGPLSSVS
ARLRLAGWHKYLTRNQIQPIAEREGDWSAMSGFQQTMQMLNEGIVPTAM
LVANDQMALGAMRAITESGLRVGADISVVGYDDTEDSSCYIPPLTTIKQ
DFRLLGQTSVDRLLQLSQGQAVKGNQLLPVSLVKRKTTLAPNTQTASPR
ALADSLMQLARQVSRLESGQ

The amino acid sequence of the tetracycline repressor from Escherichia coli is available from UNiprot (accession number B1VCFO) and is shown below as SEQ ID No. 3.

Amino acid sequence of tetracycline repressor
SEQ ID No. 3
MMSRLDKSKVINSALELLNEVGIEGLTTRKLAQKLGVEQPTLYWHVKNK
RALLDALAIEMLDRHHTHFCPLEGESWQDFLRNNAKSFRCALLSHRDGA
KVHLGTRPTEKQYETLENQLAFLCQQGFSLENALYALSAVGHFTLGCVL
EDQEHQVAKEERETPTTDSMPPLLRQAIELFDHQGAEPAFLFGLELIIC
GLEKQLKCESGS

The amino acid sequence of GAL4 is available from Uniprot (accession number P04386) and is shown below as SEQ ID No. 4.

GAL4 transcription factor
SEQ ID No. 4
MKLLSSIEQACDICRLKKLKCSKEKPKCAKCLKNNWECRYSPKTKRSPL
TRAHLTEVESRLERLEQLFLLIFPREDLDMILKMDSLQDIKALLTGLFV
QDNVNKDAVTDRLASVETDMPLTLRQHRISATSSSEESSNKGQRQLTVS
IDSAAHHDNSTIPLDFMPRDALHGFDWSEEDDMSDGLPFLKTDPNNNGF
FGDGSLLCILRSIGFKPENYTNSNVNRLPTMITDRYTLASRSTTSRLLQ
SYLNNFHPYCPIVHSPTLMMLYNNQIEIASKDQWQILFNCILAIGAWCI
EGESTDIDVFYYQNAKSHLTSKVFESGSIILVTALHLLSRYTQWRQKTN
TSYNFHSFSIRMAISLGLNRDLPSSFSDSSILEQRRRIWWSVYSWEIQL
SLLYGRSIQLSQNTISFPSSVDDVQRTTTGPTIYHGIIETARLLQVFTK
IYELDKTVTAEKSPICAKKCLMICNEIEEVSRQAPKFLQMDISTTALTN
LLKEHPWLSFTRFELKWKQLSLIIYVLRDFFTNFTQKKSQLEQDQNDHQ
SYEVKRCSIMLSDAAQRTVMSVSSYMDNHNVTPYFAWNCSYYLFNAVLV
PIKTLLSNSKSNAENNETAQLLQQINTVLMLLKKLATFKIQTCEKYIQV
LEEVCAPFLLSQCAIPLPHISYNNSNGSAIKNIVGSATIAQYPTLPEEN
VNNISVKYVSPGSVGPSPVPLKSGASFSDLVKLLSNRPPSRNSPVTIPR
STPSHRSVTPFLGQQQQLQSLVPLTPSALFGGANFNQSGNIADSSLSFT
FTNSSNGPNLITTQTNSQALSQPIASSNVHDNFMNNEITASKIDDGNNS
KPLSPGWTDQTAYNAFGITTGMFNTTTMDDVYNYLFDDEDTPPNPKKE

The Q system is derived from the qa gene cluster of Neurospora which is involved in the catabolism of quinic acid. The transcriptional activator QA-1F (QF) binds to regulatory sequences within the qa cluster and activates transcription in the absence of quinic acid.

The sequence of the transcriptional activator QF is available from Uniprot (accession number P11638) and its amino acid sequence is shown below as SEQ ID No. 5.

Amino acid sequence of quinic acid utilization
activator (QF)
SEQ ID No. 5
MPPKRKTLNAAAEANAHADGHADGNADGHVANTAASSNNARFADLTNID
TPGLGPTTTTLLVEPARSKRQRVSRACDQCRAAREKCDGIQPACFPCVS
QGRSCTYQASPKKRGVQTGYIRTLELALAWMFENVARSEDALHNLLVRD
AGQGSALLVGKDSPAAERLHARWATSRVNKSITRLLSGQAAQDPSEDGQ
SPSEDINVQDAGAKTSDFPHAPHLTFSAPKSSTAETRTLPGPVRPPISA
NTLENNLQPDGTGIGKLPPNHWRLLDIYFSYTHSWLPILEKKDMYQALY
QYSEQGSLLPSANVESGVHAELWSALALASFQAAATAASSATGPASAAH
GHDNAINPSPADISDTARKLIPLESGPFQVQHCRALLLLCLVSLGRDDW
ESAWLLVGFAVRVLLVVRTQLPPDDDRPRPRMRALLVACFIVDTIVSMR
HNVPAHLKPDDIADLPLPEDGQDQWEPWTPCEGLGGEHTMLQMLRNPAY
PLSTFNHLYGVTKLVALELLPRIRTSSQNAPLEFRSRLQQVIGHNSPFS
VFVLSQDTASAFVPTAYLTRTVYLWAAAFSEPLNEHYSHLLIETLDQYQ
KRFGTYAIPPLIPSLLDSLLALKKQSHSSERHRRHLEELFPAYSSIWPR
GGRHSNTGLQPIRQLELPPTATATASIMPHVMEQPLSTSINPVNDRFNG
IPNPTPYNSDAALDAITQTNDYGSVNTHGILSTYPPPATHLNEASVALA
PGGAPPRPPPPYVDSTTNHPPYHSNLVPMANFGYSTVDYDAMVDDLASI

EYTDAVDVDPQFMTNLGFVPGCNFSDISTYEQ

### Artifical Promoters Active in Immune Cells

Artificial promoters which function in human immune cells can be constructed by fusing the DNA-binding domain (of e.g. a prokaryotic DNA binding element) to the transactivation domain of another transcription factor functional in human immune cells. One such transactivation domain is that of herpes simplex virus virion protein 16 (VP16).

VP16 sequence
SEQ ID 6
EFPGIRRPAGIPGDLAPPTDVSLGDELHLDGEDVAMAHADALDDFDLDMLG
DGDSPGPGFTPHDSAPYGALDMADFEFEQMFTDALGIDEYGG

The LexA/VP16 fusion is functional in human cells and its sequence is shown below as SEQ ID No. 7.

Amino acid sequence of LexA/VP16 fusion
SEQ ID No. 7
MKALTARQQEVFDLIRDHISQTGMPPTRAEIAQRLGFRSPNAAEEHLKALA
RKGVIEIVSGASRGIRLLQEEEEGLPLVGRVAAGEPLLAQQHIEGHYQVDP
SLFKPNADFLLRVSGMSMKDIGIMDGDLLAVHKTQDVRNGQVVVARIDDEV
TVKRLKKQGNKVELLPENSEFKPIVVDLRQQSFTIEGLAVGVIRNGDWLEF
PGIRRPAGIPGDLAPPTDVSLGDELHLDGEDVAMAHADALDDFDLDMLGDG
DSPGPGFTPHDSAPYGALDMADFEFEQMFTDALGIDEYGG

The LacR/VP16 fusion protein is functional in human cells and its sequence is available and is shown below as SEQ ID No. 8.

Amino acid sequence of lactose repressor/VP16
fusion
SEQ ID No. 8
MVNVKPVTLYDVAEYAGVSYQTVSRVVNQASHVSAKTREKVEAAMAELNYI
PNRVAQQLAGKQSLLIGVATSSLALHAPSQIVAAIKSRADQLGASVVVSMV
ERSGVEACKAAVHNLLAQRVSGLIINYPLDDQDAIAVEAACTNVPALFLDV
SDQTPINSIIFSHEDGTRLGVEHLVALGHQQIALLAGPLSSVSARLRLAGW
HKYLTRNQIQPIAEREGDWSAMSGFQQTMQMLNEGIVPTAMLVANDQMALG
AMRAITESGLRVGADISVVGYDDTEDSSCYIPPLTTIKQDFRLLGQTSVDR
LLQLSQGQAVKGNQLLPVSLVKRKTTLAPNTQTASPRALADSLMQLARQVS
RAPPTDVSLGDELHLDGEDVAMAHADALDDFDLDMLGDGDSPGPGFTPHDS
APYGALDMADFEFEQMFTDALGIDEYGG

The tetracycline-controlled transactivator (tTA) consists of a fusion between tetracycline repressor and the transactivation domain of herpes simplex virus virion protein 16 (VP16). The amino acid sequence of tTA is shown below as SEQ ID No. 9.

Amino acid sequence of tetracycline-controlled
transactivator (tTA)
SEQ ID No. 9
MMSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVHL
GTRPTEKQYETLENQLAFLCQQGFSLENALYALSAVGHFTLGCVLEDQEHQ
VAKEERETPTTDSMPPLLRQAIELFDHQGAEPAFLFGLELIICGLEKQLKC
ESGSAYSRARTKNNYGSTIEGLLDLPDDDAPEEAGLAAPRLSFLPAGHTRR
LSTAPPTDVSLGDELHLDGEDVAMAHADALDDFDLDMLGDGDSPGPGFTPH
DSAPYGALDMADFEFEQMFTDALGIDEYGG

An artificial transcription factor can be created by fusing the DNA-binding domain of GAL4 to the transactivation domain of VP16.

The amino acid sequence of the GAL4/VP16 fusion protein is given below as SEQ ID No 10.

Amino acid sequence of GAL4/VP16
SEQ ID No. 10
MKLLSSIEQACDICRLKKLKCSKEKPKCAKCLKNNWECRYSPKTKRSPLTR
AHLTEVESRLERLEQLFLLIFPREDLDMILKMDSLQDIKALLTGLFVQDNV
NKDAVTDRLASVETDMPLTLRQHRISATSSSEESSNKGQRQLTVSPEFPGI
WAPPTDVSLGDELHLDGEDVAMAHADALDDFDLDMLGDGDSPGPGFTPHDS
APYGALDMADFEFEQMFTDALGIDEYGG

The DNA-binding specificity of the transcriptional activator QF from Neurospora can be redirected to alternate sites by exchanging its DNA-binding domain for that of another transcription factor such as LexA.

The amino acid sequence of the LexA/QF artificial transcription factor is shown below as SEQ ID No. 11.

Amino acid sequence of LexA/QF fusion protein
SEQ ID No. 11
MPPKKKRKVEDPMKALTARQQEVFDLIRDHISQTGMPPTRAEIAQRLGFRS
PNAAEEHLKALARKGVIEIVSGASRGIRLLQEEEEGLPLVGRVAAGEPLLA
QQHIEGHYQVDPSLFKPNADFLLRVSGMSMKDIGIMDGDLLAVHKTQDVRN
GQVVVARIDDEVTVKRLKKQGNKVELLPENSEFKPIVVDLRQQSFTIEGLA
VGVIRNGDWLGRQLELPPTATATASIMPHVMEQPLSTSINPVNDRFNGIPN
PTPYNSDAALDAITQTNDYGSVNTHGILSTYPPPATHLNEASVALAPGGAP
PRPPPPYVDSTTNHPPYHSNLVPMANFGYSTVDYDAMVDDLASIEYTDAVD
VDPQFMTNLGFVPGCNFSDINTYEQ

### Non-Endogenous Transcription Factors

The transcription factor may be “non-endogenous” in the sense that it is not usually expressed in the cell type (i.e. an immune cell) which is transduced with the kit of vectors.

The transcription factor may be usually expressed in a non-immune cell.

The transcription factor may be derivable, for example, from neuronal cells or muscle cells.

Neuronal cell specific transcription factors include those that drive expression of the nestin promoter (see next section). The nestin promoter is driven by the POU family of transcription factors 1 and includes Brain-1 (Brn-1; also known as POU domain, class 3, transcription factor 3 [POU3F3]); and Brain-2 (Brn-2; also known as POU domain, class 3, transcription factor 2 [POU3F2]).

The DNA binding sequence of Brain-1 is 5′-ATTTGCAT-3′ (SEQ ID No. 12). The amino acid sequence is available from Uniprot (P20264) and is shown as SEQ ID No. 13 below.

Brain-1 amino acid sequence
SEQ ID No. 13
MATAASNPYLPGNSLLAAGSIVHSDAAGAGGGGGGGGGGGGGGAGGGGGGM
QPGSAAVTSGAYRGDPSSVKMVQSDFMQGAMAASNGGHMLSHAHQWVTALP
HAAAAAAAAAAAAVEASSPWSGSAVGMAGSPQQPPQPPPPPPQGPDVKGGA
GRDDLHAGTALHHRGPPHLGPPPPPPHQGHPGGWGAAAAAAAAAAAAAAAA
HLPSMAGGQQPPPQSLLYSQPGGFTVNGMLSAPPGPGGGGGGAGGGAQSLV
HPGLVRGDTPELAEHHHHHHHHAHPHPPHPHHAQGPPHHGGGGGGAGPGLN
SHDPHSDEDTPTSDDLEQFAKQFKQRRIKLGFTQADVGLALGTLYGNVFSQ
TTICRFEALQLSFKNMCKLKPLLNKWLEEADSSTGSPTSIDKIAAQGRKRK
KRTSIEVSVKGALESHFLKCPKPSAQEITNLADSLQLEKEVVRVWFCNRRQ
KEKRMTPPGIQQQTPDDVYSQVGTVSADTPPPHHGLQTSVQ

The DNA binding sequence recognised by Brain-2 consists of two half sites (GCAT) and (TAAT), separated by a non-conserved spacer region of 0, 2, or 3 nucleotides. The amino acid sequence of Brain-2 is available from Uniprot (Accession Number: P20265) and is shown below as SEQ ID No. 14.

Brain-2 amino acid sequence
SEQ ID No. 14
MATAASNHYSLLTSSASIVHAEPPGGMQQGAGGYREAQSLVQGDYGALQSN
GHPLSHAHQWITALSHGGGGGGGGGGGGGGGGGGGGGDGSPWSTSPLGQPD
IKPSVVVQQGGRGDELHGPGALQQQHQQQQQQQQQQQQQQQQQQQQQRPPH
LVHHAANHHPGPGAWRSAAAAAHLPPSMGASNGGLLYSQPSFTVNGMLGAG
GQPAGLHHHGLRDAHDEPHHADHHPHPHSHPHQQPPPPPPPQGPPGHPGAH
HDPHSDEDTPTSDDLEQFAKQFKQRRIKLGFTQADVGLALGTLYGNVFSQT
TICRFEALQLSFKNMCKLKPLLNKWLEEADSSSGSPTSIDKIAAQGRKRKK
RTSIEVSVKGALESHFLKCPKPSAQEITSLADSLQLEKEVVRVWFCNRRQK
EKRMTPPGGTLPGAEDVYGGSRDTPPHHGVQTPVQ

Other neuronal-cell specific transcription factors include those which drive transcription from the glial fibrillary acidic protein (GFAP) promoter (see next section). The GFAP promoter is regulated by several transcription factors, including nuclear factor 1C (NFIC) and 1X (NFIX).

The DNA binding site of NFIC is 5′-TTGGCXXXXXGCCAA-3′ (SEQ ID No. 15). The amino acid sequence of NFIC is available from Uniprot (Accession No. P08651) and is shown below as SEQ ID No. 16.

Human Nuclear factor 1 C amino acid sequence
SEQ ID No. 16
MYSSPLCLTQDEFHPFIEALLPHVRAFAYTWFNLQARKRKYFKKHEKRMSK
DEERAVKDELLGEKPEVKQKWASRLLAKLRKDIRPECREDFVLSITGKKAP
GCVLSNPDQKGKMRRIDCLRQADKVWRLDLVMVILFKGIPLESTDGERLVK
AAQCGHPVLCVQPHHIGVAVKELDLYLAYFVRERDAEQSGSPRTGMGSDQE
DSKPITLDTTDFQESFVTSGVFSVTELIQVSRTPVVTGTGPNFSLGELQGH
LAYDLNPASTGLRRTLPSTSSSGSKRHKSGSMEEDVDTSPGGDYYTSPSSP
TSSSRNWTEDMEGGISSPVKKTEMDKSPFNSPSPQDSPRLSSFTQHHRPVI
AVHSGIARSPHPSSALHFPTTSILPQTASTYFPHTAIRYPPHLNPQDPLKD
LVSLACDPASQQPGPLNGSGQLKMPSHCLSAQMLAPPPPGLPRLALPPATK
PATTSEGGATSPTSPSYSPPDTSPANRSFVGLGPRDPAGIYQAQSWYLG

The DNA binding site of NFIC is 5′-TTGGCXXXXXGCCAA-3′ (SEQ ID No. 17). The amino acid sequence of NFIX is available from Uniprot (Accession No. Q14938) and is shown below as SEQ ID No. 18.

Human Nuclear factor 1X amino acid sequence
SEQ ID No. 18
MYSPYCLTQDEFHPFIEALLPHVRAFSYTWFNLQARKRKYFKKHEKRMSKD
EERAVKDELLGEKPEIKQKWASRLLAKLRKDIRPEFREDFVLTITGKKPPC
CVLSNPDQKGKIRRIDCLRQADKVWRLDLVMVILFKGIPLESTDGERLYKS
PQCSNPGLCVQPHHIGVTIKELDLYLAYFVHTPESGQSDSSNQQGDADIKP
LPNGHLSFQDCFVTSGVWNVTELVRVSQTPVATASGPNFSLADLESPSYYN
INQVTLGRRSITSPPSTSTTKRPKSIDDSEMESPVDDVFYPGTGRSPAAGS
SQSSGWPNDVDAGPASLKKSGKLDFCSALSSQGSSPRMAFTHHPLPVLAGV
RPGSPRATASALHFPSTSIIQQSSPYFTHPTIRYHHHHGQDSLKEFVQFVC
SDGSGQATGQPNGSGQGKVPGSFLLPPPPPVARPVPLPMPDSKSTSTAPDG
AALTPPSPSFATTGASSANRFVSIGPRDGNFLNIPQQSQSWFL

Muscle cell specific transcription factors include those that drive expression from the myogenin (myog) promoter; the murine muscle creatine kinase promoter; or the muscle creatine kinase (MCK) promoter/α-myosin heavy chain enhancer hybrid (referred to as MHCK7). DNA-binding sites present in the myog, MCK and MHCK7 promoter regions include those for the transcription factors: myoblast determination protein 1 (MyoD1); myocyte-specific enhancer factor 2A (MEF2A) 5; and Krueppel-like factor 3 (KLF3) 7.

MyoD is the master regulator of muscle-specific genes and has been implicated in regulating the expression of 1,953 genes. MyoD binds to the promoter region of myogenin 5: the DNA binding site of MyoD is 5′-CANNTG-3′ (SEQ ID No. 19). The amino acid sequence of human myoblast determination protein 1 (MyoD1) is available from Uniprot (Accession No. P15172) and is shown below as SEQ ID No. 20.

Human myoblast determination protein 1 (MyoD1)
amino acid sequence
SEQ ID No. 20
MELLSPPLRDVDLTAPDGSLCSFATTDDFYDDPCFDSPDLRFFEDLDPRLM
HVGALLKPEEHSHFPAAVHPAPGAREDEHVRAPSGHHQAGRCLLWACKACK
RKTTNADRRKAATMRERRRLSKVNEAFETLKRCTSSNPNQRLPKVEILRNA
IRYIEGLQALLRDQDAAPPGAAAAFYAPGPLPPGRGGEHYSGDSDASSPRS
NCSDGMMDYSGPPSGARRRNCYEGAYYNEAPSEPRPGKSAAVSSLDCLSSI
VERISTESPAAPALLLADVPSESPPRRQEAAAPSEGESSGDPTQSPDAAPQ
CPAGANPNPIYQVL

Myocyte-specific enhancer factor 2A (MEF2A) binds not only to the myogenin promoter region, but also to the MCK promoter. The DNA binding site of MEF2A is 5′-YTA[AT]4TAR-3′ (SEQ ID No. 21). The amino acid sequence for human myocyte-specific enhancer factor 2A (MEF2A) amino acid sequence is available from Uniprot (Accession No. Q02078) and is shown below as SEQ ID No. 22.

Human myocyte-specific enhancer factor 2A (MEF2A)
SEQ ID No. 22
MGRKKIQITRIMDERNRQVTFTKRKFGLMKKAYELSVLCDCEIALIIFNSS
NKLFQYASTDMDKVLLKYTEYNEPHESRTNSDIVEALNKKEHRGCDSPDPD
TSYVLTPHTEEKYKKINEEFDNMMRNHKIAPGLPPQNFSMSVTVPVTSPNA
LSYTNPGSSLVSPSLAASSTLTDSSMLSPPQTTLHRNVSPGAPQRPPSTGN
AGGMLSTTDLTVPNGAGSSPVGNGFVNSRASPNLIGATGANSLGKVMPTKS
PPPPGGGNLGMNSRKPDLRVVIPPSSKGMMPPLSEEEELELNTQRISSSQA
TQPLATPVVSVTTPSLPPQGLVYSAMPTAYNTDYSLTSADLSALQGFNSPG
MLSLGQVSAWQQHHLGQAALSSLVAGGQLSQGSNLSINTNQNISIKSEPIS
PPRDRMTPSGFQQQQQQQQQQQPPPPPQPQPQPPQPQPRQEMGRSPVDSLS
SSSSSYDGSDREDPRGDFHSPIVLGRPPNTEDRESPSVKRMRMDAWV

KLF3 is a known positive regulator of the MCK promoter, and although KLF3 lacks a transactivation domain (TAD), it is thought to exert its regulatory function through its interaction with serum response factor (SRF), a ubiquitously expressed transcription factor. The DNA-binding site of KLF3 is 5′-C(A/C)CACCC-3′ (SEQ ID No. 23). The amino acid sequence of human Krueppel-like factor 3 (KLF3) is available from Uniprot (Accession No. P57682) and is shown below as SEQ ID No. 24.

Human Krueppel-like factor 3 (KLF3) amino acid
sequence
SEQ ID No. 24
MLMFDPVPVKQEAMDPVSVSYPSNYMESMKPNKYGVIYSTPLPEKFFQTPE
GLSHGIQMEPVDLTVNKRSSPPSAGNSPSSLKFPSSHRRASPGLSMPSSSP
PIKKYSPPSPGVQPFGVPLSMPPVMAAALSRHGIRSPGILPVIQPVVVQPV
PFMYTSHLQQPLMVSLSEEMENSSSSMQVPVIESYEKPISQKKIKIEPGIE
PQRTDYYPEEMSPPLMNSVSPPQALLQENHPSVIVQPGKRPLPVESPDTQR
KRRIHRCDYDGCNKVYTKSSHLKAHRRTHTGEKPYKCTWEGCTWKFARSDE
LTRHFRKHTGIKPFQCPDCDRSFSRSDHLALHRKRHMLV

### Custom Zinc Finger-Based Artificial Transcription Factors

Artificial transcription factors can be constructed using zinc finger domains, which are short domain of approximately 30 amino acids that have a similar structure but vary in binding specificity. A single zinc finger domain is capable of recognising 3 base pairs of DNA and by splicing multiple zinc fingers together using amino acid linker sequences it is possible to generate a DNA-binding protein with a defined specificity. By fusing an array of zinc finger domains to a suitable transactivation domain (e.g. VP16), it is possible to construct an artificial transcription factor capable of recognising a pre-defined DNA sequence present in an artificial upstream activating sequence (UAS).

### Custom TAL-Based Artificial Transcriptional Factors

Transcription activation-like (TAL) effectors are programmable transcription factors that offer the ability to construct artificial promoters. A TAL effector comprises a central repeat region of a variable number of 34 amino acid repeats, two C-terminal nuclear localisation sequences and a C-terminal transactivation domain 21. The repeat region dictates the DNA-binding specificity of the TAL effector, with each repeat recognising a single base pair of DNA (meaning that the number of repeats in the TAL effector defines how many base pairs it will recognise) and hyper-variability in residues 12 and 13 of the 34 amino acid determines which nucleotide it will recognise. As the DNA-binding specificity of TAL effectors is programmable, it is possible to construct artificial transcription factors recognising cognate DNA sequences in promoter regions by fusion with an appropriate transcription factor.

### CRISPR-Based Artificial Transcriptional Factors

Many bacteria possess an adaptive immune system that protects them from invading bacteriophages. This system is encoded on a locus comprising an array of clustered regularly interspaced short palindromic repeat (CRISPR) sequences and associated proteins (Cas proteins) that are involved in processing and utilising RNA transcribed from the CRISPR array to recognise and cleave the DNA of invading viruses. Cleavage of the viral DNA is carried out by the endonuclease Cas9, which is guided to a specific site in the viral genome by an RNA derived from the CRISPR array.

As Cas9 is targeted to a specific DNA sequence via a guide RNA (gRNA), which can be easily synthesized in vitro or in vivo, the CRISPR/Cas9 system can be repurposed for modifying genomic DNA sequences in eukaryotes. Further adaptations of the CRISPR/Cas9 system, by rendering Cas9 catalytically inactive and coupling it to either a transcriptional activator or repressor, have enabled its use in controlling gene expression.

Systems utilising catalytically inactive Cas9 (dCas9) coupled to a transactivation domain (TAD), such as VP16, have been described to drive the expression of endogenous genes in yeast and human cells. These systems can be easily adapted to generate artificial transcriptional circuits using defined promoter sequences and complementary RNAs to guide the dCas9/TAD fusion protein to them and initiate gene transcription.

The amino acid sequence of dCas9/VP64 is shown below as SEQ ID No. 25. In this sequence, the large T-antigen (Uniprot; P0307) SV40 nuclear localization sequences are shown in bold; the linker sequence is in italics; and the VP64 transactivation domain is underlined.

dCas9/VP64
SEQ ID No. 25
MKKKRKVEASDKKYSIGLAIGTNSVGWAVITDEYKVPSKKFKVLGNTDRHS
IKKNLIGALLFDSGETAEATRLKRTARRRYTRRKNRICYLQEIFSNEMAKV
DDSFFHRLEESFLVEEDKKHERHPIFGNIVDEVAYHEKYPTIHLRKKLVDS
TDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLG
LTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKDK
DFLDNEENEDILEDIVLTLTLFEDREMIEERLKTYAHLFDDKVMKQLKRRR
YTGWGRLSRKLINGIRDKQSGKTILDFLKSDGFANRNFMQLIHDDSLTFKE
DIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQN
EKLYLYYLQNGRDMYVDQELDINRLSDYDVDAIVPQSFLKDDSIDNKVLTR
SDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELLG
ITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGD
PKKKRKVDALDDFDLDMLGSDALDDFDLDMLGSDALDDFDLDMLGSDALDD
FDLDMLYID

The first cassette in the transcriptional circuit may express the dCas9/TAD fusion protein and contain a U6 promoter driving the transcription of a gRNA targeting the artificial promoter region in the second cassette of the transcriptional circuit. Candidate promoter regions that can be used to drive expression of the second cassette include a tissue-specific promoter, such as the MHCK7 hybrid promoter described below, or an artificial transcriptional circuit based on the GAL4 UAS, TetR, and LacI promoters, which have been employed previously in eukaryotes to control the expression of exogenous genes.

Alternatively, an entirely artificial promoter sequence that does not share homology with any other sequence in the human genome can be constructed. The gRNAs required to target the Cas9/TAD fusion to the artificial promoter regions can be designed using freely available design tools such as CRISPOR (http://crispor.tefor.net/) to ensure that they do not exhibit off-targeting and initiate unwanted expression of endogenous genes. The expression of both dCas9/TAD and the gRNA from the first retroviral cassette then drives transcription from the artificial promoter in the second cassette.

### Promoters

Eukaryotic expression cassettes use promoter sequences to drive transgene expression. Promoter sequences function by their recognition by transcription factors which upon binding to the promoter sequences initiation transcription by engaging RNA polymerase to the transcriptional start site.

The vector may comprise a constitutively active promoter, such as the immediate early cytomegalovirus (CMV) promoter sequence, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, or a human gene promoter such as the actin promoter, the myosin promoter, the elongation factor-la promoter, the hemoglobin promoter, or the creatine kinase promoter.

Alternatively, the vector may comprise an inducible promoter. The use of an inducible promoter provides a molecular switch capable of turning on expression of the polynucleotide sequence to which it is operatively linked when expression is desired, or turning off the expression when expression is not desired. Examples of inducible promoters include a metallothionine promoter, a glucocorticoid promoter, a progesterone promoter, and a tetracycline-regulated promoter.

Alternatively, the promoter may only be active in the presence of a transcription factor provided by another vector.

The promoter sequence may be activated by an artificial transcription factor. The promoter sequence may be activated by an artificial transcription factor which comprises a guide RNA. The transcription factor may, for example, comprise the RNA-guided endonuclease Cas9 (dCas9) fused to a transcription activation domain (TAD) (see previous section).

Candidate promoter regions that can be used to drive expression of the second vector include a tissue-specific promoter, such as the MHCK7 hybrid promoter described below, or an artificial transcriptional circuit based on the GAL4 upstream activation sequence, TetR, and/or LacI promoters, which have been employed in eukaryotes to control the expression of exogenous genes. Alternatively, an entirely artificial promoter sequence that does not share homology with any other sequence in the human genome can be constructed.

The nucleic acid sequence of a 5-repeat GAL4 upstream activating sequence is shown as SEQ ID No. 26. In this sequence, GAL4 DNA binding sequences (5′-CGG-N11-CCG-3′ -SEQ ID No. 46) are underlined.

GAL4 5x upstream activating sequence
SEQ ID No. 26
CGGAGTACTGTCCTCCGAGSCGGAGTACTGTCCTCCGACTCGAGCGGAGTA
CTGTCCTCCGATCGGAGTACTGTCCTCCGCGAATTCCGGAGTACTGTCCTC
CG

The Tet response element comprises seven repeats of the tetracycline operator (tetO) sequence. The nucleic acid sequence of the Tet response element (TRE) is shown below as SEQ ID No. 27. In this sequence, tetO sequences are underlined.

Tet response element (TRE)
SEQ ID No. 27
TCCCTATCAGTGATAGAGAAAAGTGAAAGTCGAGTTTACCACTCCCTATC
AGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGT
GAAAGTCGAGTTTACCACTCCCTATCAGTGATAGAGAAAAGTGAAAGTCG
AGTTTACCACTCCCTATCAGTGATAGAGAAAAGTGAAAGTCGAGTTTACC
ACTCCCTATCAGTGATAGAGA

The nucleic acid sequence of the LacI promoter sequence is shown below as SEQ ID No. 28.

LacI promoter sequence
SEQ ID No. 28
GACACCATCGAATGGCGCAAAACCTTTCGCGGTATGGCATGATAGCGCCC
GGAAGAGAGTCAATTCAGGGTGGTGAAT

The promotor sequence may be an artificial promoter sequence designed to be recognised by an artificial transcription factor such as transcription activation-like (TAL) effector (see previous section).

TAL effectors are programmable transcription factors that offer the ability to construct artificial promoters. As the DNA-binding specificity of TAL effectors is programmable, it is possible to construct artificial transcription factors recognising cognate DNA sequences in promoter regions. It is also possible to construct artificial promoter sequences for use with known or artificial transcription factors using the TAL effector technology.

The promoter sequence may be from a natural gene, for example from a prokaryotic or eukaryotic organism. Where the promoter is derived from a human gene, in order to avoid background transcription, the promoter may be activated by a “non-endogenous” transcription factor, i.e. a transcription factor which is not usually expressed in the host cell type. The promoter sequence may be “non-endogenous” in the sense that it is derived from a gene which is not normally expressed in the host cell. The promoter sequence may be derived from a gene which is usually expressed in a non-immune cell. The promoter sequence may be tissue-specific for non-immune tissue. Potential tissue-specific promoters include those controlling the expression of muscle-specific or neuronal-specific genes.

### Neuronal-Specific Promoters

Nestin is an intermediate-filament protein that is expressed by stem cells during neurogenesis and is downregulated as the cells differentiate into neurons.

The promoter region derived from intron 2 of the nestin gene has been widely used to drive transgene expression in conditional knock-out and knock-in mouse models. Expression of the nestin promoter is driven by the POU family of transcription factors (see above). The nucleic acid sequence of Murine nestin intron 2 is available from GenBank (Accession No. AY438043.1) and is shown below as SEQ ID No. 29.

Murine nestin intron 2
SEQ ID No. 29
GGTCTGAAAAGGATTTGGAGAAGGGGAGCTGAATTCATTTGCTTTTGTCT
GTTACCAGCTCTGGGGGCAGAGAGAGAGCCATCCCCTGGGAACAGCCTGA
GAATTCCCACTTCCCCTGAGGAGCCCTCCCTTCTTAGGCCCTCCAGATGG
TAGTGTGGACAAAAGGCAATAATTAGCATGAGAATCGGCCTCCCTCCCAG
AGGATGAGGTCATCGGCCTTGGCCTTGGGTGGGGAGGCGGAGACTGATCT
GAGGAGT

Glial fibrillary acidic protein (GFAP) is an intermediate filament protein whose expression is restricted to astrocytes. The promoter region of GFAP has been used to drive the expression of transgenes in mouse models and is a suitable promoter to form part of a transcriptional circuit. The GFAP promoter is regulated by several transcription factors, including nuclear factor 1C (NFIC) and 1X (NFIX), as mentioned above. The nucleic acid sequence for the GFAP promoter is shown below as SEQ Id No. 30.

Glial Fibrillary Acidic Protein (GFAP) Promoter
sequence
SEQ ID No. 30
ACATATCCTGGTGTGGAGTAGGGGACGCTGCTCTGACAGAGGCTCGGGGG
CCTGAGCTGGCTCTGTGAGCTGGGGAGGAGGCAGACAGCCAGGCCTTGTC
TGCAAGCAGACCTGGCAGCATTGGGCTGGCCGCCCCCCAGGGCCTCCTCT
TCATGCCCAGTGAATGACTCACCTTGGCACAGACACAATGTTCGGGGTGG
GCACAGTGCCTGCTTCCCGCCGCACCCCAGCCCCCCTCAAATGCCTTCCG
AGAAGCCCATTGAGCAGGGAGCTCTCCCCATAGCTGGGCTGCGGCCCAAC
CCCACCCCCTCAGGCTATGCCAGGGGGTGTTGCCAGGGGCACCCGGGCAT
CGCCAGTCTAGCCCACTCCTTCATAAAGCCCTCGCATCCCAGGAGCGAGC
AGAGCCAGAGCAGGTTGGAGAGGAGACGCATCACCTCCGCTGCTCGCGGG
GATCCTCTAGAGTCGACGGATCCGGGGAATTCCCCAGTCTCAGGATCCAC
CATGGGG

A transcriptional circuit can be constructed by placing a transgene encoding a neuronal specific transcription factor on the first cassette and its cognate promote sequence on the second cassette to drive transgene expression.

### Muscle-Specific Promoter Regions

Several muscle-specific promoters are suitable for use in driving the expression of the genes encoded on the second cassette, and include the myogenin (myog) promoter, murine muscle creatine kinase promoter, and the muscle creatine kinase (MCK) promoter/α-myosin heavy chain enhancer hybrid (referred to as MHCK7). The latter promoter exhibits low expression in non-muscle cells and has been shown to be inactive in dendritic cell lines. The muscle-specific promoter regions possess binding sites for transcription factors, which function in a co-ordinated manner to drive gene transcription. DNA-binding sites present in the myog, MCK and MHCK7 promoter regions include those for the transcription factors myoblast determination protein 1 (MyoD1), myocyte-specific enhancer factor 2A (MEF2A) 5 and Krueppel-like factor 3 (KLF3) 7 (see previous section).

A transcription circuit can be built by placing a muscle-specific transcription factor (MyoD, MEF2 or KLF3) on the first cassette and a compatible promoter region (myogenin, MCK or MHCK7) on the second cassette to drive expression of the transgenes encoded on it.

The nucleic acid sequence of murine myogenin promoter sequence is available from GenBank (Accession No. X71910.1) and is shown as SEQ ID No. 31

murine myogenin promoter
SEQ ID No. 31
ATCCACTGGAAACGTCTTGATGTGCAGCAACAGCTTAGAGGGGGGCTCAG
GTTTCTGTGGCGTTGGCTATATTTATCTCTGGGTTCATGCCAGCAGGGAG
GGTTTAAATGGCACCCAGCAGTTGGTGTGAGGGGCTGCGGGAGCTTGGGG
G

The nucleic acid sequence of murine muscle creatine kinase promoter is available from GenBank (Accession No AF188002.1) and is shown below as SEQ ID No. 32.

murine muscle creatine kinase promoter
SEQ ID No. 32
CCACTACGGGTCTAGGCTGCCCATGTAAGGAGGCAAGGCCTGGGGACACC
CGAGATGCCTGGTTATAATTAACCCAGACATGTGGCTGCCCCCCCCCCCC
CAACACCTGCTGCCTCTAAAAATAACCCTGTCCCTGGTGGAT

The nucleic acid sequences of murine muscle creatine kinase and α-myosin heavy chain (MHCK7) promoter are available from GenBank (Accession numbers AF188002.1 and U71441.1 respectively). The combined sequence is shown below as SEQ ID No. 33, in which the murine α-myosin heavy chain enhancer sequence is in normal text and the murine muscle creatine kinase promoter region is underlined.

Murine muscle creatine kinase/α-myosin heavy chain
(MHCK7) promoter
SEQ ID No. 33
CCTTCAGATTAAAAATAACTGAGGTAAGGGCCTGGGTAGGGGAGGTGGTG
TGAGACGCTCCTGTCTCTCCTCTATCTGCCCATCGGCCCTTTGGGGAGGA
GGAATGTGCCCAAGGACTAAAAAAAGGCCATGGAGCCAGAGGGGCGAGGG
CAACAGACCTTTCATGGGCAAACCTTGGGGCCCTGCTGTCTAGCATGCCC
CACTACGGGTCTAGGCTGCCCATGTAAGGAGGCAAGGCCTGGGGACACCC
GAGATGCCTGGTTATAATTAACCCAGACATGTGGCTGCCCCCCCCCCCCC
AACACCTGCTGCCTCTAAAAATAACCCTGTCCCTGGTGGATCCCCTGCAT
GCGAAGATCTTCGAACAAGGCTGTGGGGGACTGAGGGCAGGCTGTAACAG
GCTTGGGGGCCAGGGCTTATACGTGCCTGGGACTCCCAAAGTATTACTGT
TCCATGTTCCCGGCGAAGGGCCAGCTGTCCCCCGCCAGCTAGACTCAGCA
CTTAGTTTAGGAACCAGTGAGCAAGTCAGCCCTTGGGGCAGCCCATACAA
GGCCATGGGGCTGGGCAAGCTGCACGCCTGGGTCCGGGGTGGGCACGGTG
CCCGGGCAACGAGCTGAAAGCTCATCTGCTCTCAGGGGCCCCTCCCTGGG
GACAGCCCCTCCTGGCTAGTCACACCCTGTAGGCTCCTCTATATAACCCA
GGGGCACAGGGGCTGCCCTCATTCTACCACCACCTCCACAGCACAGACAG
ACACTCAGGAGCCAGCCAGCC

### Cassettes

The vectors in the kit of the present invention comprise a combination of genetic elements in the form of a cassette.

The first vector in the kit of vectors of the invention comprises a first transgene and a nucleotide sequence encoding a transcription factor.

The first vector may comprise a cassette having the general structure:
TG1-coexpr-TF or
TF-coexpr-TG1
in which:
TG1 is a first transgene
Coexpr is a nucleotide sequence enabling co-expression of the two flanking polypeptides (in this case TF and the polypeptide encoded by TG1)
TF is a nucleotide sequence encoding the transcription factor

The second vector in the kit of vectors of the invention comprises a second transgene under the control of a promoter which is activated by the transcription factor.

The second vector may comprise a cassette having the general structure:
pT-TG2
in which pT is a promoter activated by the transcription factor; and
TG2 is a second transgene.

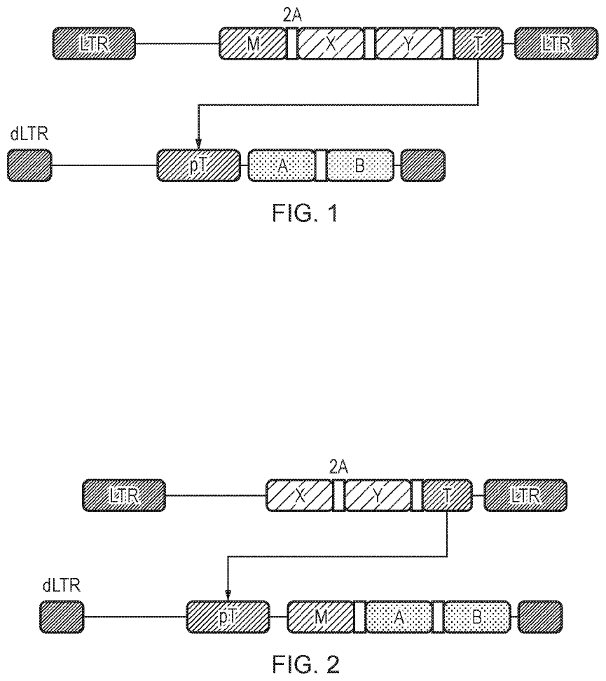

FIG. 1 illustrates an arrangement having a first vector with a marked primary cassette and second vector with a dependent secondary cassette. The primary cassette is driven by a constitutively active promoter and comprises a marker gene, a first transgene and a nucleotide sequence encoding a transcription factor. The secondary cassette is driven by a promoter sequence dependent on the transcription factor supplied by the primary cassette and comprises a second transgene. In this setup, after double-transduction the culture could contain immune cells which have not been transduced by the primary cassette and hence express no transgenes or immune cells which express the primary cassette only, or immune cells which express the primary and secondary cassettes. If the primary cassette also encodes for a marker gene, cells can be selected so that immune cells which do not express transgenes are removed.

The primary cassette for this arrangement may comprise one the following general structures:
M-coexpr-TG1-coexpr-TF
M-coexpr-TF-coexpr-TG1
TG1-coexpr-M-coexpr-TF
TG1-coexpr-TF-coexpr-M
TF-coexpr-M-coexpr-TG1
TF-coexpr-TG1-coexpr-M

In which:
M is a marker gene and coexpr, TG1 and TF are as defined above. The secondary cassette for this arrangement may comprise the structure pT-TG2, as defined above.

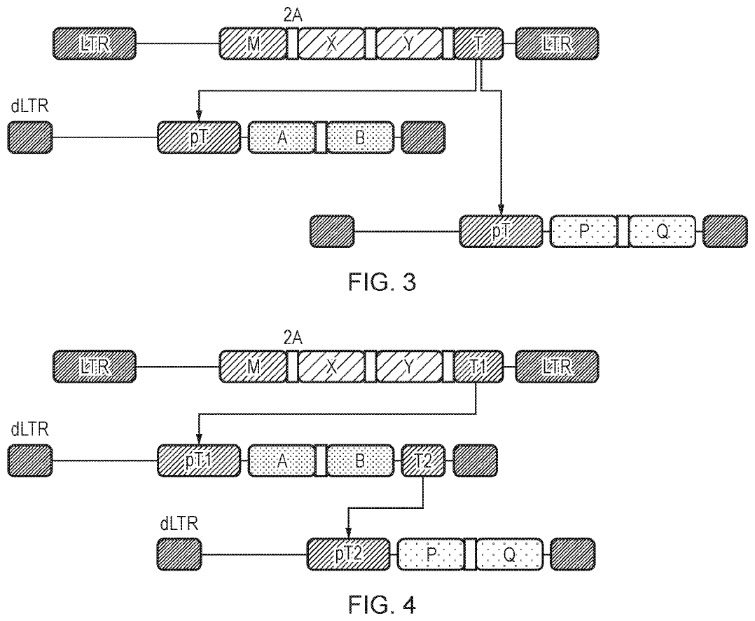

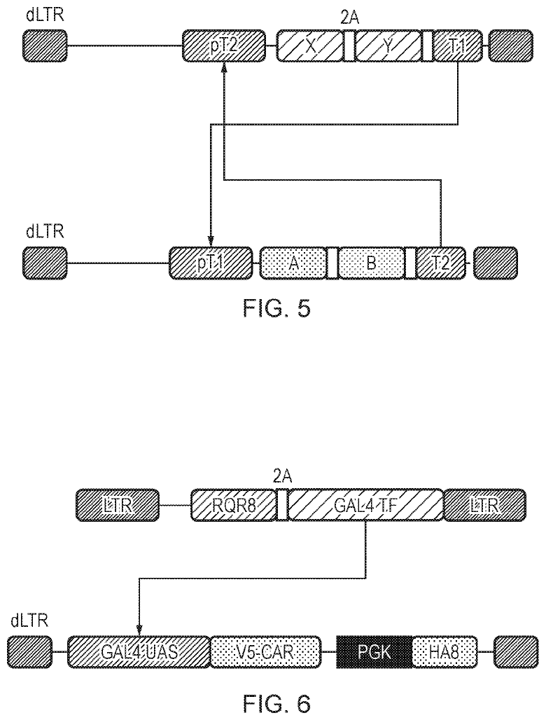

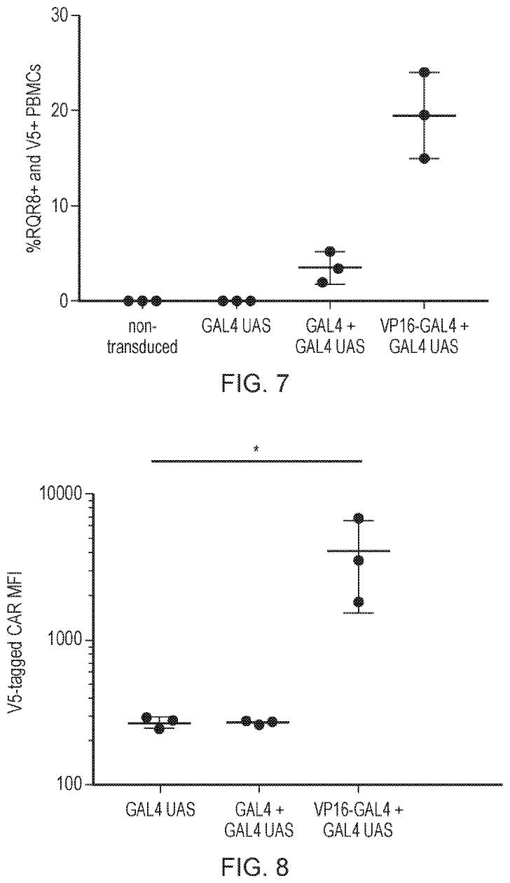

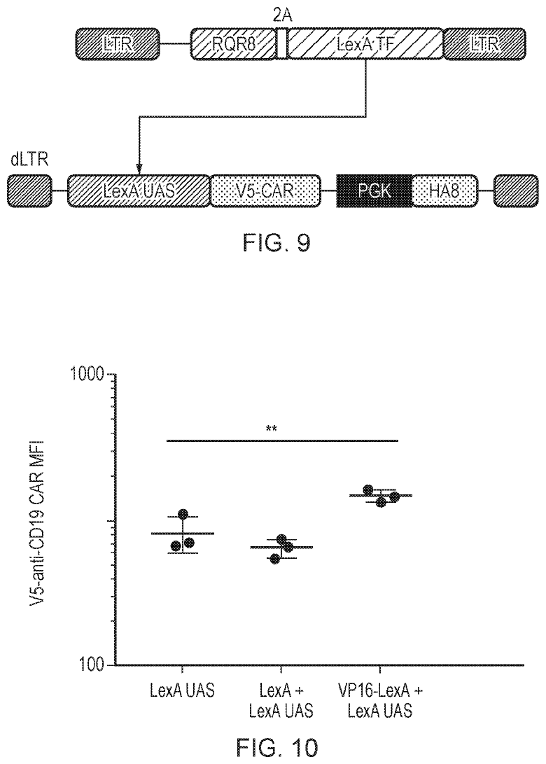

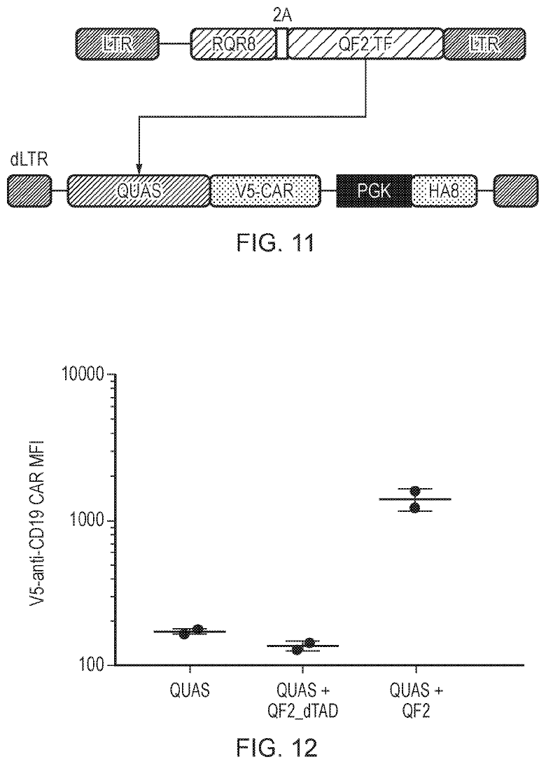

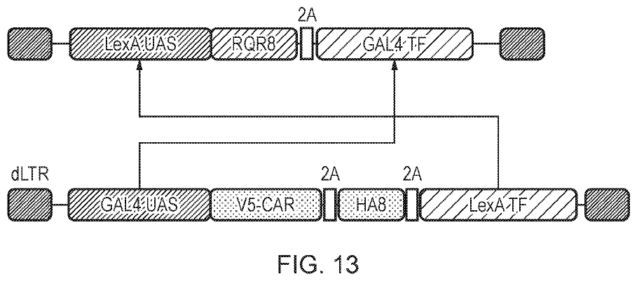

FIG. 3 illustrates a similar scenario, but one in which there are multiple dependent cassettes. In this the third cassette may have the general formula pT-TG3, in which TG3 is a third transgene and pT is as defined above.

FIG. 2 illustrates an arrangement having a first vector with a primary cassette and second vector with a marked dependent secondary cassette. The primary cassette is driven by a constitutively active promoter and comprises a first transgene and a nucleotide sequence encoding a transcription factor. The secondary cassette is driven by a promoter sequence dependent on the transcription factor supplied by the primary cassette and comprises a marker gene and a second transgene. In this setup, after transduction with both vectors, the culture will contain immune cells which express neither cassette, the primary cassette only or the primary and secondary cassette. In this case sorting on the marker gene which is expressed in the secondary cassette results in the selection of double-expressing cells only.

The first vector may comprise a cassette having the general structure as defined above:
TG1-coexpr-TF or
TF-coexpr-TG1

The second vector may comprise a cassette having the general structure:
pT-TG2-coexpr-M or
pT-M-coexpr-TG2
in which pT, TG2, coexpr and M are as defined above.

FIG. 4 illustrates an arrangement having three or more vectors with iterative dependent cassettes. The primary cassette is driven by a constitutively active promoter and comprises a marker gene, a first transgene and a nucleotide sequence encoding a first transcription factor. The second cassette is driven by a promoter sequence dependent on the first transcription factor supplied by the primary cassette and comprises a second transgene and a nucleotide sequence encoding a second transcription factor. The tertiary cassette is driven by a promoter sequence dependent on the second transcription factor supplied by the secondary cassette and comprises a third transgene. In this scenario, an iterative dependence of expression is required down the chains of expression cassettes. If a marker is expressed by the last expression cassette, sorting will select cells which express all cassettes.

The primary cassette may have a general structure as described above for the arrangement shown in FIG. 1. The secondary cassette may have the general structure:
pT1-TG2-coexpr-TF2, or
pT1-TF2-coexpr-TG2
in which pT1 is a promoter activated by the first transcription factor, TF2 is a nucleotide sequence encoding the second transcription factor and TG2 and coexpr are as defined above.

The tertiary cassette may have the general structure:
pT2-TG3
in which pT2 is a promoter activated by the second transcription factor and TG3 is a third transgene.

FIG. 5 illustrates an arrangement having a two or more vectors with circular dependent cassettes. The primary cassette comprises a first transgene and is driven by a promoter sequence dependent on a second transcription factor supplied by the second cassette The secondary cassette comprises a second transgene and is driven by a promoter sequence dependent on a first transcription factor supplied by the first cassette. The two cassettes are therefore inter-dependent i.e. the expression from one is dependent on a transcription factor expressed by the other and vice versa. In this case, if cells are transduced with only one cassette, expression is silent. If a cell is transduced with both cassettes, a small leak of expression present in all such systems is be amplified such that both cassettes would be expressed.

### Transgene

In the kit of vectors of the present invention, one or more vectors may express a transgene, i.e. a nucleotide sequence which transcribes or encodes an entity of interest (EOI).

The kit of vectors is for transducing an immune cell with multiple transgenes. The term “multiple” means two or more, as the kit of vectors comprise at least a first and a second transgene. The kit may include 3, 4, 5, 6, 7 or more transgenes. The transgene may be split between first and second and/or further vectors. A vector may express multiple transgenes by using a co-expression sequence such as an IRES or a self-cleaving peptide (see below).

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Transgenes or nucleic acids according to the invention may comprise DNA or RNA. They may be single-stranded or double-stranded. They may also be polynucleotides which include within them synthetic or modified nucleotides.

The transgene may encode a marker protein or it may enable selection of transduced cells for example by providing an antibiotic or drug-resistance gene to the cell.

The transgene may encode an activatory or inhibitory chimeric antigen receptor (CAR) or a T cell receptor (TCR).

The transgene may encode a signal transduction modifying protein.

The transgene may enhance proliferation, engraftment and/or survival of immune cells. For example, the transgene may encode a cytokine, cytokine receptor, chimeric cytokine receptor, dominant-negative SHP-1 or SHP-2, dominant negative TGFbeta of TGFbeta receptor, or a constitutively active or inducible JAK or STAT (see below).

The transgene may or allow pharmacological control of a CAR or TCR. For example, it may encode a signalling component for use in an inducible or disruptible CAR system, or encode a dampener of T-cell mediated signalling.

The transgene may encode a suicide switch.

When two vectors A and B are transduced into a cell, there are four possible outcomes:
a) no successful transduction with A or B
b) transduction with A but not B
c) transduction with B but not A
d) transduction with A and B

A kit of vectors of the invention may comprise:
(i) a first vector which comprises a first transgene under the control of a constitutively active promoter and a nucleotide sequence encoding a transcription factor and; and
(ii) a second vector which comprises a second transgene under the control of a promoter dependent on the transcription factor supplied by the first vector.

In this case, transduction with the first vector alone will result in expression of the first transgene by the cell, however transduction with the second vector alone will not result in expression of the second transgene alone. In this system it is not possible to get expression of the second transgene without expression of the first transgene.

This is useful in situations where a CAR or TCR is co-expressed with an auxiliary gene, for example a gene which affects the activity of the CAR/TCR or the CAR/TCR expressing cell, and where it is desirable to avoid expression of the auxiliary gene without the CAR/TCR.

In this embodiment, the “first transgene”, produced by the first vector and under the control of a constitutively active promoter, may encode a CAR or TCR; and the “second transgene”, produced by the second vector and under the control of a promoter dependent on the transcription factor supplied by the first vector, may encode a factor which affects the activity of the CAR/TCR or the CAR/TCR expressing cell.

The second transgene may, for example, encode a cytokine, a cytokine receptor, a dominant negative TGF beta or TGF beta receptor, a signal transduction modifying protein, constitutively active JAK/STAT, an inhibitor CAR, a suicide switch or a dampener (see below).

The second vector may also comprise a marker gene. Expression of the marker gene indictates successful transduction with both the first and second vector (or first, second and third vectors).

### Marker

One or more of the vectors in the kit may comprise a marker gene, which makes a cell expressing the gene detectable or selectable. It allows for identification of successful transduction.

For example, a marker gene may encode a marker protein which is detectable by cell surface expression or another property such as fluorescence or radio-labelling.

A marker protein may be derivable from CD34. CD34 is a cell surface glycoprotein and functions as a cell-cell adhesion factor. It also mediates the attachment of stem cells to bone marrow extracellular matrix or directly to stromal cells.

CD34 is not expressed by terminally differentiated haematopoietic lineages, so it is an ideal marker for modified T-cells.

CD34-expressing cells may be readily identified and isolated using the Miltenyi CliniMACS magnetic cell selection system, which is a commonly used reagent for clinical stem cell isolation. The CliniMACS CD34 selection system utilises the QBEnd10 monoclonal antibody to achieve cellular selection.

The QBEnd10-binding epitope from within the CD34 antigen has the amino acid sequence shown as SEQ ID No. 34.

(SEQ ID NO. 34)
ELPTQGTFSNVSTNVS

The marker protein may comprise SEQ ID No. 34.

A compact sort-suicide gene comprising the QBEnd10-binding epitope is described in WO2013/153391.

Another example of a marker protein is a truncated version of the epidermal growth factor receptor (tEGFR) that lacks the EGF binding and intracellular signaling domains. Cell surface tEGFR can be detected by biotnylated anti-EGFR (Erbitux) monoclonal antibodies.

Various antibiotic resistance genes have been described which are useful markers for transduction. For example, the bacterial neomycin and hygromycin phosphotransferase genes confer resistance to G418 and hygromycin, respectively; a mutant mouse dihydrofolate reductase gene (dhfr*) confers resistance to methotrexate; the bacterial gpt gene which cells to grow in medium containing mycophenolic acid, xanthine, and aminopterin; the bacterial hisD gene which allows cells to grow in medium without histidine but containing histidinol; the multidrug resistance gene (mdr) which confers resistance to a variety of drugs; and the bacterial genes which confer resistance to puromycin or phleomycin.

### Chimeric Antigen Receptor (CAR)

One or more of the vector(s) of the kit of the invention may comprise a transgene encoding a chimeric antigen receptor (CAR).

A classical CAR is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of lgG1. More compact spacers can suffice e.g. the stalk from CD8a and even just the lgG1 hinge alone, depending on the antigen. A trans-membrane domain anchors the protein in the cell membrane and connects the spacer to the endodomain.

Early CAR designs had endodomains derived from the intracellular parts of either the y chain of the FcER1 or CD37. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD37 results in second generation receptors which can transmit an activating and co-stimulatory signal simultaneously after antigen recognition. The co-stimulatory domain most commonly used is that of CD28. This supplies the most potent co-stimulatory signal-namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general formula:
Signal peptide-antigen binding domain-spacer domain-transmembrane domain-intracellular T cell signaling domain (endodomain).

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell. The antigen binding domain may be based on a natural ligand of the antigen.

The antigen binding domain may comprise an affinity peptide from a combinatorial library or a de novo designed affinity protein/peptide.

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

The transmembrane domain may be derived from CD28, which gives good receptor stability.

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28; and/or
(iii) a domain which transmits a survival signal, for example a TNF receptor family endodomain such as OX-40 or 4-1BB.

A number of systems have been described in which the antigen recognition portion is on a separate molecule from the signal transmission portion, such as those described in WO015/150771; WO2016/124930 and WO2016/030691. The CAR expressed by the cell of the present invention may therefore comprise an antigen-binding component comprising an antigen-binding domain and a transmembrane domain; which is capable of interacting with a separate intracellular signalling component comprising a signalling domain. The vector of the invention may express a CAR signalling system comprising such an antigen-binding component and intracellular signalling component.

The CAR may comprise a signal peptide so that when it is expressed inside a cell, the nascent protein is directed to the endoplasmic reticulum and subsequently to the cell surface, where it is expressed. The signal peptide may be at the amino terminus of the molecule.

### T-Cell Receptor

One or more of the vector(s) of the kit of the invention may comprise a transgene encoding a T-cell receptor (TCR).

The TCR is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (a) chain and a beta (B) chain (encoded by TRA and TRB, respectively), whereas in 5% of T cells the TCR consists of gamma and delta (y/8) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vector. For example the genes for engineered TCRs may be reintroduced into autologous T cells and transferred back into patients for T cell adoptive therapies.

### Signal Transduction Modifying Protein

WO2016/193696 describes various fusion proteins and truncated proteins which modulate the signalling pathways following immune cell activation.

The transgene may for example, encode one of the following signal transduction modifying proteins:
(i) a truncated protein which comprises an SH2 domain from a protein which binds a phosphorylated immunoreceptor tyrosine-based activation motif (ITAM), but lacks a kinase domain;
(ii) a truncated protein which comprises an SH2 domain from a protein which binds a phosphorylated immunoreceptor tyrosine-based inhibition motif (ITIM) but lacks a phosphatase domain;
(iii) a fusion protein which comprises (a) an SH2 domain from a protein which binds a phosphorylated immunoreceptor tyrosine-based activation motif (ITAM) or from a protein which binds a phosphorylated immunoreceptor tyrosine-based inhibition motif (ITIM); and (ii) a heterologous domain.

The signal transduction modifying protein may be a truncated protein which comprises a ZAP70 SH2 domain but lacks a ZAP70 kinase domain.

The signal transduction modifying protein may be a fusion protein which comprises (i) an SH2 domain from a protein which binds a phosphorylated immunoreceptor tyrosine-based activation motif (ITAM); and (ii) a phosphatase domain.

The fusion protein may, for example, comprise a ZAP70 SH2 domain, a PTPN6 or an SHP-2 phosphatase domain.

The signal transduction modifying protein may be a fusion protein which comprises (i) an SH2 domain from a protein which binds a phosphorylated immunoreceptor tyrosine-based inhibition motif (ITIM); and (ii) a kinase domain.

The fusion protein may comprise an SH2 domain from PTPN6 or SHP-2.

The fusion protein may comprise a Zap70 kinase domain

The fusion protein may comprise an AKT or JAK kinase domain.

The signal transduction modifying protein may be a fusion protein which comprises (i) an SH2 domain from a protein which binds a phosphorylated immunoreceptor tyrosine-based activation motif (ITAM) or from a protein which binds a phosphorylated immunoreceptor tyrosine-based inhibition motif (ITIM); and (ii) a heterologous signalling domain.

The fusion protein may comprise an SH2 domain from ZAP70, PTPN6 or SHP-2.

The heterologous signalling domain may be from a signalling molecule which is not usually activated by an ITAM or ITIM containing receptor.

The heterologous signalling domain may be a co-stimulatory domain. In this respect, the fusion protein may comprise a CD28, OX40 or 41BB co-stimulatory domain.

The heterologous signalling domain may be an inhibitory domain. In this respect, the inhibitory domain may be or comprise the endodomain of CD148 or CD45. Alternatively, the heterologous signalling domain is or comprises the endodomain of ICOS, CD27, BTLA, CD30, GITR or HVEM.

The signal transduction modifying protein may be a fusion protein which comprises (i) an SH2 domain from a protein which binds a phosphorylated immunoreceptor tyrosine-based activation motif (ITAM); and (ii) an ITAM-containing domain.

The fusion protein may comprises a ZAP70 SH2 domain.

The ITAM-containing domain may be or comprise the endodomain of CD3-Zeta.

The signal transduction modifying protein may be a fusion protein which comprises (i) an SH2 domain from a protein which binds a phosphorylated immunoreceptor tyrosine-based inhibition motif (ITIM); and (ii) an ITIM-containing domain.

The fusion protein may comprise an SH2 domain from PTPN6 or SHP-2.

The ITIM-containing domain may be or comprise the endodomain from PD1, PDCD1, BTLA4, LILRB1, LAIR1, CTLA4, KIR2DL1, KIR2DL4, KIR2DL5, KIR3DL1 or KIR3DL3.

### SRC Homology Region 2 Domain-Containing Phosphatase-1 (SHP-1)

The signal transduction modifying protein may be a modified form of SHP-1, which lacks a functional phosphatase domain. For example, the signal transduction modifying protein may be a truncated protein which comprises an SHP-1 SH2 but lacks a SHP-1 phosphatase domain.

SHP-1 is also known as tyrosine-protein phosphatase non-receptor type 6 (PTPN6). It is a member of the protein tyrosine phosphatase family.

The N-terminal region of SHP-1 contains two tandem SH2 domains which mediate the interaction of SHP-1 and its substrates. The C-terminal region contains a tyrosine-protein phosphatase domain.

SHP-1 is capable of binding to, and propagating signals from, a number of inhibitory immune receptors or ITIM containing receptors. Examples of such receptors include, but are not limited to, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350.

The signal transduction modifying protein may comprise or consist of a SHP-1 SH2 domain. In this respect, the STMP may comprise or consist of the sequence shown as SEQ ID NO: 35.

SHP-1 SH2 complete domain
(SEQ ID NO: 35)
MVRWFHRDLSGLDAETLLKGRGVHGSFLARPSRKNQGDFSLSVRVGDQVT
HIRIQNSGDFYDLYGGEKFATLTELVEYYTQQQGVLQDRDGTIIHLKYPL
NCSDPTSERWYHGHMSGGQAETLLQAKGEPWTFLVRESLSQPGDFVLSVL
SDQPKAGPGSPLRVTHIKVMCEGGRYTVGGLETFDSLTDLVEHFKKTGIE
EASGAFVYLRQPYY

SHP-1 has two SH2 domains at the N-terminal end of the sequence, at residues 4-100 and 110-213 of the sequence shown as SEQ ID No. 35. The STMP of the invention may therefore comprise one or both of the sequences shown as SEQ ID No. 36 and 37.

SHP-1 SH2 1
(SEQ ID NO: 36)
WFHRDLSGLDAETLLKGRGVHGSFLARPSRKNQGDFSLSVRVGDQVTHIR
IQNSGDFYDLYGGEKFATLTELVEYYTQQQGVLQDRDGTIIHLKYPL
SHP-1 SH2 2
(SEQ ID No. 37)
WYHGHMSGGQAETLLQAKGEPWTFLVRESLSQPGDFVLSVLSDQPKAGPG
SPLRVTHIKVMCEGGRYTVGGLETFDSLTDLVEHFKKTGIEEASGAFVYL
RQPY

The STMP may comprise a variant of SEQ ID NO: 35, 36 or 37 having at least 80, 85, 90, 95, 98 or 99% sequence identity, provided that the variant sequence is a SH2 domain sequence capable of binding a pITIM domain. For example, the variant sequence may be capable of binding to the phosphorylated tyrosine residues in the cytoplasmic tail of PD1, PDCD1, BTLA4, LILRB1, LAIR1, CTLA4, KIR2DL1, KIR2DL4, KIR2DL5, KIR3DL1 or KIR3DL3. The variant sequence may be the equivalent sequence to of SEQ ID NO: 35, 36 or 37 when derived from isoform 2, 3 or 4 of SHP-1.

### SHP-2

The signal transduction modifying protein may be a modified form of SHP-2, which lacks a functional phosphatase domain. For example, the signal transduction modifying protein may be a truncated protein which comprises an SHP-2 SH2 but lacks a SHP-2 phosphatase domain.

SHP-2, also known as PTPN11, PTP-1D and PTP-2C, is also a member of the protein tyrosine phosphatase (PTP) family. Like SHP-1, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1.

The signal transduction modifying protein may comprise or consist of a SHP-2 SH2 domain. In this respect, the STMP may comprise or consist of the first SH2 domain of SHP-2, for example comprising amino acids 6-102 of SEQ ID NO. 5 or the second SH2 domain of SHP-2, for example comprising amino acids 112-216 of SHP-2. The STMP may comprise or consist of the sequence shown as SEQ ID NO: 38, 39 or 40. The STMP may comprise a variant of SEQ ID NO: 38, 39 or 40 having at least 80, 85, 90, 95, 98 or 99% sequence identity, provided that the variant sequence is a SH2 domain sequence capable of binding a pITIM domain. For example, the variant sequence may be capable of binding to the phosphorylated tyrosine residues in the cytoplasmic tail of PD1, PDCD1, BTLA4, LILRB1, LAIR1, CTLA4, KIR2DL1, KIR2DL4, KIR2DL5, KIR3DL1 or KIR3DL3. The variant sequence may be the equivalent sequence to of SEQ ID NO: 38, 39 or 40 when derived from isoform 2 or 3 of SHP-2.

SHP-2 first SH2 domain
(SEQ ID NO: 38)
WFHPNITGVEAENLLLTRGVDGSFLARPSKSNPGDFTLSVRRNGAVTHIK
IQNTGDYYDLYGGEKFATLAELVQYYMEHHGQLKEKNGDVIELKYPL
SHP-2 second SH2 domain
(SEQ ID No. 39)
WFHGHLSGKEAEKLLTEKGKHGSFLVRESQSHPGDFVLSVRTGDDKGESN
DGKSKVTHVMIRCQELKYDVGGGEREDSLTDLVEHYKKNPMVETLGTVLQ
LKQPL
SHP-2 both SH2 domains
(SEQ ID No. 40)
WFHPNITGVEAENLLLTRGVDGSFLARPSKSNPGDFTLSVRRNGAVTHIK
IQNTGDYYDLYGGEKFATLAELVQYYMEHHGQLKEKNGDVIELKYPLNCA
DPTSERWFHGHLSGKEAEKLLTEKGKHGSFLVRESQSHPGDFVLSVRTGD
DKGESNDGKSKVTHVMIRCQELKYDVGGGERFDSLTDLVEHYKKNPMVET
LGTVLQLKQPL

The expression of a signal transduction modifying protein which comprises a modified or truncated SHP-1 or SHP-2 with an inactive or removed phosphatase domain can help counteract a hostile microenvironment for example due to PD-L1 overexpression. In solid cancers such as prostate cancer, the microenvironment may be particularly hostile due to PDL1 over-expression; in this case a decision might be made to include a truncated SHP-1 or SHP-2 as a transgene in the kit of vectors.

### TGF Beta/TGF Beta Receptor

The transgene may encode a protein capable of enhancing proliferation of cells such as T cells. For example, the transgene may encode a mutant version of transforming growth factor beta which inhibits TGFbeta secretion or action.

Lopez et al ((1992) Mol. Cell Biol. 12:1674-9) describe dominant negative mutants of TGFbeta 1 comprising mutations in the precursor domain.

Alternatively that transgene may encode a dominant-negative version of a TGF beta receptor, such as a truncated TGF beta receptor

A truncated TGF beta receptor 2 may comprise the sequence shown as SEQ ID No. 41.

SEQ ID No. 41
MGWSCIILFLVATATGVHSTIPPHVQKSVNNDMIVTDNNGAVKFPQLCKF
CDVRFSTCDNQKSCMSNCSITSICEKPQEVCVAVWRKNDENITLETVCHD
PKLPYHDFILEDAASPKCIMKEKKKPGETFFMCSCSSDECNDNIIFSEEY
NTSNPDLLLVIFQVTGISLLPPLGVAISVIIIFYCYRVNRQQKLSS

In this sequence:
The leader sequence from murine Ig heavy chain V region 102 (Uniprot; P01750) is in bold.
TGFβRII ectodomain is in normal text (Uniprot; P37173)
TGFβRII transmembrane domain is underlined.
TGFβRII truncated cytoplasmic domain is in italics.

The expression of a dominant negative TGF beta or TGF beta receptor can help counteract a hostile microenvironment for example due to TGFbeta overexpression. In this case a decision might be made to include expression of a dominant-negative TGFbeta or TGFBeta receptor as a transgene.

### Dampener

In an alternative embodiment, the transgene may encode a phosphatase “damper” which causes dephosphorylation of a CAR or TCR endodomain, raising the threshold to activation in certain transcriptional states.

The dampener may be a membrane-tethered signal-dampening component (SDC) comprising a signal-dampening domain (SDD).

The SDD may be capable of inhibiting the intracellular signalling domain of the CAR.

The SDD may comprise a phosphatase domain capable of dephosphorylating immunoreceptor tyrosine-based activation motifs (ITAMs), for example the endodomain of CD148 or CD45 or the phosphatase domain of SHP-1 or SHP-2.

The SDD may comprise an immunoreceptor tyrosine-based inhibition motif (ITIM), for example the SDD may comprise an endodomain from one of the following inhibitory receptors: PD1, BTLA, 2B4, CTLA-4, GP49B, Lair-1, Pir-B, PECAM-1, CD22, Siglec 7, Siglec 9, KLRG1, ILT2, CD94-NKG2A and CD5.

The SDD may inhibits a Src protein kinase, such as Lck. The SDD may comprise the kinase domain of CSK.

The membrane-tethered SDC may, for example, comprise a transmembrane domain or a myristoylation sequence.

The dampener may be inducible, for example, by the addition of a small molecule. Various inducible dampener systems are described in GB1707780.1, GB1707781.9 and GB1707783.5.

### Inhibitory Car

The transgene may encode an inhibitory CAR, i.e. a CAR which comprises an inhibitory endodomain. Inhibitory CARs are described in WO2015/075470. The inhibitory endodomain may comprise a protein-tyrosine phosphatase (PTP), such as the PTP domain from SHP-1 or SHP-2.

Alternatively, the inhibitory endodomain may comprise an ITIM (Immunoreceptor Tyrosine-based Inhibition motif) containing endodomain such as that from CD22, LAIR-1, the Killer inhibitory receptor family (KIR), LILRB1, CTLA4, PD-1, BTLA etc. When phosphorylated, ITIMs recruits endogenous PTPN6 through its SH2 domain. If co-localised with an ITAM containing endodomain, dephosphorylation occurs and the activating CAR or TCR is inhibited.

Alternatively, the inhibitory CAR may comprise a phosphatase domain capable of dephosphorylating immunoreceptor tyrosine-based activation motifs (ITAMs), for example the endodomain of CD148 or CD45 or the phosphatase domain of SHP-1 or SHP-2.

### Cytokines, Cytokine Receptors, Cytokine Signalling Domains and Chimeric Cytokine Receptors

The transgene may encode a cytokine such as IL-2, IL-7, IL-12 or IL-15.

The transgene may encode a cytokine receptor.

The transgene may encode a protein comprising a cytokine receptor endodomain.

The transgene may encode a chimeric cytokine receptor which comprises an exodomain which binds to a target antigen, such as a tumour secreted antigen, a tumour antigen or a chemokine and a cytokine receptor endodomain. Chimeric cytokine receptors are described in more detail in WO2017/029512.

### JAK/STAT

The transgene may encode a constitutively active or inducible Signal Transducer and Activator of Transcription (STAT) molecule or a constitutively active or inducible Janus Kinase (JAK) molecule. Examples of such molecules are given in GB1714718.2.

The cassette may comprise a first transgene encoding a first STAT polypeptide comprising a first dimerizing domain (DD) and a second transgene encoding a second STAT polypeptide comprising a second DD, which specifically binds to the first DD. Binding of the first and second DDs may be inducible with an agent.

A constitutively active STAT molecule may comprise a gain-of-function mutation.

### Suicide Switch

The transgene may be suicide-gene, which is a genetically encoded mechanism which allows selective destruction of adoptively transferred T-cells in the face of unacceptable toxicity. Two suicide-genes have been tested in clinical studies: Herpes Simplex Virus thymidine kinase (HSV-TK) and inducible caspase 9 (iCasp9).

The herpes simplex virus I-derived thymidine kinase (HSV-TK) gene has been used as an in vivo suicide switch in donor T-cell infusions to treat recurrent malignancy and Epstein Barr virus (EBV) lymphoproliferation after hemopoietic stem cell transplantation.

The activation mechanism behind Caspase 9 was exploited in the original iCasp9 molecule. All that is needed for Caspase 9 to become activated, is overcoming the energic barrier for Caspase 9 to homodimerize. The homodimer undergoes a conformational change and the proteolytic domain of one of a pair of dimers becomes active. Physiologically, this occurs by binding of the CARD domain of Caspase 9 to APAF-1. In iCasp9, the APAF-1 domain is replaced with a modified FKBP12 which has been mutated to selectively bind a chemical inducer of dimerization (CID). Presence of the CID results in homodimerization and activation. iCasp9 is based on a modified human caspase 9 fused to a human FK506 binding protein (FKBP) (Straathof et al (2005) Blood 105:4247-4254). It enables conditional dimerization in the presence of a small molecule CID, known as AP1903.

An alternative caspase-based suicide gene is described in WO2016/135470. The transgene may encode a suicide gene having the general structure: FRB-FKBP12-dCasp9 or FKBP12-FRB-dCasp9. For example, the transgene may encode the amino acid sequence shown as SEQ ID No. 42.

(FRB-FKBP12-L3-dCasp9)
SEQ ID No. 42
<-----------------------FRB-----------------------
MASRILWHEMWHEGLEEASRLYFGERNVKGMFEVLEPLHAMMERGPQTLK
---------------------------FRB------------------->
ETSFNQAYGRDLMEAQEWCRKYMKSGNVKDLLQAWDLYYHVFRRISKLEY
<L1-><--FKBP12------------------------FKBP12------
SGGGSLEGVQVETISPGDGRTFPKRGQTCVVHYTGMLEDGKKFDSSRDRN
--------------------------------------------------
KPFKFMLGKQEVIRGWEEGVAQMSVGQRAKLTISPDYAYGATGHPGIIPP
-------------><------L3------><--dCasp9-----------
HATLVFDVELLKLESGGGGSGGGGSGGGGSGVDGFGDVGALESLRGNADL
--------------dCasp9------------------------------
AYILSMEPCGHCLIINNVNFCRESGLRTRTGSNIDCEKLRRRFSSLHFMV
------------------------dCasp9--------------------
EVKGDLTAKKMVLALLELAQQDHGALDCCVVVILSHGCQASHLQFPGAVY
----------------------------------dCasp9----------
GTDGCPVSVEKIVNIFNGTSCPSLGGKPKLFFIQACGGEQKDHGFEVAST
--------------------------------------------dCasp9
SPEDESPGSNPEPDATPFQEGLRTFDQLDAISSLPTPSDIFVSYSTFPGF
--------------------------------------------dCasp9
VSWRDPKSGSWYVETLDDIFEQWAHSEDLQSLLLRVANAVSVKGIYKQMP
---------------->
GCFNFLRKKLFFKTSAS

The transgene may encode the sort-suicide gene known as RQR8 which described in WO2013/153391, which comprises the amino acid sequence shown as SEQ ID No. 43

SEQ ID No. 43
CPYSNPSLCSGGGGSELPTQGTFSNVSTNVSPAKPTTTACPYSNPSLCSG
GGGSPAPRPPTPAPTIASQPLSLRPEACRPAAGGAVHTRGLDFACDIYIW
APLAGTCGVLLLSLVITLYCNHRNRRRVCKCPRPVV

Cells comprising this suicide gene may be deleted by the administration of Rituximab.

The transgene may encode a suicide switch which comprises a multi-spanning transmembrane protein, such as CD20, fused to a FAS endodomain. When expressed at the cell surface, the multi-spanning transmembrane protein binds an extracellular ligand, leading to activation of the FAS endodomain. These systems are described in more detail in WO2016/174408.

### Co-Expression

In the cassettes and vectors of the invention, the nucleic acid sequences may be connected by sequences enabling co-expression of the transgene sequences as separate polypeptides. For example, the nucleic acid may encode a cleavage site between two transgenes; or two cleavage sites, enabling the production of three transgenes as discrete polypeptides. The cleavage site may be self-cleaving, such that when the compound polypeptide is produced, it is immediately cleaved into the separate components without the need for any external cleavage activity.

Various self-cleaving sites are known, including the Foot-and-Mouth disease virus (FMDV) 2a self-cleaving peptide, which may have one of the following sequences:

SEQ ID NO: 44
RAEGRGSLLTCGDVEENPGP.
or
SEQ ID NO: 45
QCTNYALLKLAGDVESNPGP

The co-expression sequence may alternatively be an internal ribosome entry sequence (IRES) or an internal promoter.

### Cell

The present invention provides a cell which comprises a transgene and a nucleotide sequence encoding a non-endogenous transcription factor, wherein expression of the transgene is dependent upon expression of the non-endogenous transcription factor.

The present invention provides a cell transfected or transduced with a kit of vectors of the invention.

The cell may be a cytolytic immune cell.

Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class Il molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Cells of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, the cells may be derived from ex vivo differentiation of inducible progenitor cells or embryonic progenitor cells to, for example, T cells. Alternatively, an immortalized cell line which retains its lytic function and could act as a therapeutic may be used.

### Method for Making Cell or Cell Composition

The invention also provides a method for making a cell which comprises that step of transducing or transfecting a cell with a kit of vectors of the invention.

The cell may be a cell isolated from a subject. The cell may be transfected or transduced in vitro or ex vivo.

Successful transduction or transfection with both or all of the vectors in the kit may be determined by detecting expression of the marker gene, or by selection based on expression of the marker gene.

The invention also provides a method for making a cell composition expressing a kit of vectors of the invention, which comprises the following steps:
(i) transducing or transfecting a cell with a kit of vectors as described herein wherein one of the vectors comprises a marker gene; and
(ii) selecting cells which express the marker gene wherein expression of the marker gene by a cell indicates that the comprises both or all of the vectors in the kit.

The cell(s) is/are transduced or transfected ex vivo.

The invention also a method for selecting cells transduced with both or all of the vectors of the first aspect of the invention which comprises the step of selecting cells based on the expression of a marker gene.

### Composition

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The cells of the present invention may be capable of killing target cells, such as cancer cells.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

The invention will now be further described by way of Examples, which are meant to serve to assist one of ordinary skill in the art in carrying out the invention and are not intended in any way to limit the scope of the invention.

### EXAMPLES

### Example 1

### Design and Testing of a Kit of Two Vectors Where Expression of a Transgene From one Vector is Dependent on Expression of a Transcription Factor by the Other Vector

A primary cassette is generated using a retroviral transfer vector where expression is driven by the long terminal repeat of Monkey Moloney leukemia virus (MoMLV). The transgene of the primary cassette consists of the marker gene RQR8 followed by a FMD 2A like peptide and a CAR targeting GD2 followed by a further 2A peptide and a GAL4/VP16 transcription factor. A second cassette is generated using a self-inactivating retroviral transfer vector where expression is driven by a promoter comprising a GAL4 5x upstream activating sequence. The transgene consists of a second marker gene-truncated NGFR and a truncated SHP2. T-cells are transduced with either primary or the secondary cassette or both together. Non-transduced and transduced T-cells are analysed by flow-cytometry after staining for both marker genes.

### Example 2

### Design and Testing of a Transcriptional Circuit Using a GAL4 System

The GAL4 system utilises the DNA-binding domain of the yeast transcription factor GAL4, which induces the expression of genes involved in metabolising galactose to glucose. In the presence of glucose the GAL4 transcription factor is repressed and this results in the cessation of expression of the GAL4-responsive genes. The DNA sequence recognised by the GAL4 transcription factor and its functional domains (DNA-binding domain and transcriptional activation domain) have been well defined and exploited in the yeast two-hybrid system, which identifies protein-protein interactions.

The GAL4 transcriptional-dependent circuit utilises a chimeric GAL4 transcription factor consisting of the transaction domain (TAD) of the herpes simplex viral protein 16 (VP-16) fused to the DNA-binding domain (DBD) of GAL4 and a GAL4 UAS promoter containing five copies of the GAL4 DNA binding site. The VP16-GAL4 chimeric transcription factor and the GAL4 UAS promoter are present in separate retroviral vectors. This restricts transcriptional activity to cells co-transduced with both retroviruses, because in the absence of one of the components there is no transcription from the GAL4 UAS promoter.

### Constructs

The sequences encoding the chimeric VP16-GAL4 transcription factor or the GAL4 DNA-binding domain alone were cloned into the retroviral vector SFGmR downstream of the sort selection marker RQR8 (constructs AU47529 and AU47530, respectively). A self-cleaving 2A peptide sequence was placed between RQR8 and the GAL transcription factor to enable expression of both polypeptides.

To enable expression from the GAL4 UAS without interference transcriptional interference from the viral LTRs, the promoter sequence was cloned into the self-inactivating (SIN) retroviral vector pSuper upstream of a V5-tagged anti-CD19 CAR (a second generation CAR with anti-CD19 CAT19 scFv fused to the human CD8a stalk and transmembrane domains and 4-1BB and CD37 signalling domains). Within the pSuper SIN retroviral construct there is a human phosphoglycerate kinase (PGK) promoter that drives the expression of a cell surface marker consisting of a HA epitope presented on a human CD8a stalk (HA8), which enables detection of transduced cells (Table 1 and FIG. 6).

TABLE 1
Description of GAL4 system constructs

| Number | Plasmid name | Description |
|---|---|---|
| AU47529 | SFGmR.RQR8-2A-VP16-GAL4 | Retroviral construct expressing RQR8 |
| | | sort selection marker and active VP16- |
| | | GAL4 transcription factor |
| AU47530 | SFGmR.RQR8-2A-GAL4 | Control retroviral construct expressing |
| | | RQR8 sort selection marker and |
| | | inactive GAL4 transcription factor |
| | | consisting of DBD alone |
| AU47531 | pSuperRetroW.RheoRE.V5- | Self-inactivating (SIN) retroviral vector |
| | aCD19_CAT19-CD8STK-CD8TM- | containing GAL4 UAS driving the |
| | 41BBz.PGK.HA8 | expression of a V5-tagged anti-CD19 |
| | | CAR. An internal PGK promoter drives |
| | | the expression of an HA8 marker (HA |
| | | epitope presented on a human CD8a |
| | | stalk). |

### Methods

Retroviral supernatant was prepared by transfecting 293T cells with the SFGmR or pSuper retroviral constructs and a mixture of packaging constructs (gag/pol and the glycoprotein RDF114). Activated human peripheral blood mononuclear cells (PBMCs) were transduced with retroviral particles generated from the genome vectors described above and flow cytometry carried out on stained cells 72 hours after transduction. Staining was carried out using Alexa-488 conjugated anti-HA epitope, PE-conjugated anti-CD34 QBend10, and APC conjugated anti-V5 epitope antibodies. The anti-CD34 and anti-HA antibodies detected the RQR8 sort selection and HA8 markers, respectively, expressed on the surface of transduced cells. The V5 epitope antibody detected the V5-tagged anti-CD19 CAR, whose expression was driven from the UAS promoter in cells co-transduced with both the transcription factor and promoter constructs.

Results

Peripheral blood mononuclear cells were stained with antibodies recognising epitopes present on the RQR8 and HA8 markers and the V5-tagged CAR and analysed by flow cytometry to detect transduced cells. Expression of RQR8 (driven by the retroviral LTR) was readily detectable, with between 53 to 65% of the singularly transduced cells expressing the marker. Co-transduced PBMCs were shown to express both the RQR8 and HA8 markers.

Flow cytometric analysis of PBMCs for expression of the V5-tagged showed that there was a a mean 15-fold increase in the level of expression of the V5-tagged CAR on PBMCs co-transduced with the GAL4 UAS construct and the transcriptionally active VP16-GAL4 transcription factor, with 24% of cells being V5+(FIGS. 7 and 8). These results indicate that expression of the V5-tagged CAR was restricted to PBMCs co transduced with the GAL4 UAS promoter and the transcriptionally active VP16-GAL4 chimeric transcription factor. This means that the expression of CARs and auxiliary modules can be restricted by separating the components on to two retroviral constructs and co-transducing PBMCs.

### Example 3

### Design and Testing of a Transcriptional Circuit Using a LexA System

The LexA system utilises the LexA transcription factor from E. coli that regulates the expression of genes involved in DNA repair (SOS response). LexA recognises a 22 base pair DNA sequence referred to as the LexA operator sequence.

### Constructs

Similar to the GAL4 system, the LexA transcriptional dependent circuit consisted of a LexA UAS cloned into a SIN retroviral construct that contained the V5-tagged anti-CD19 CAR. The SIN retroviral construct also contained a human phosphoglycerate kinase (PGK) promoter that drives the expression of a cell surface marker consisting of an HA epitope presented on a human CD8a stalk (HA8), which enables detection of transduced cells. Transcription from the LexA UAS is driven by a chimeric transcription factor consisting of the transactivation domain of VP16 fused to the DNA-binding domain of LexA from E. coli (Table 2 and FIG. 9).

TABLE 2
LexA constructs

| Number | Plasmid name | Description |
|---|---|---|
| AU47534 | SFGmR.RQR8-2A-LexA | Retroviral construct expressing RQR8 |
| | | sort selection marker and active VP16- |
| | | LexA transcription factor |
| AU47533 | SFGmR.RQR8-2A-VP16-LexA | Control retroviral construct expressing |
| | | RQR8 sort selection marker and |
| | | inactive LexA transcription factor |
| | | consisting of DBD alone |
| AU49052 | pSERS11.LexA_UAS.V5- | Self-inactivating (SIN) retroviral vector |
| | aCD19_CAT19-CD8STK-CD8TM- | containing LexA UAS driving the |
| | 41BBz.PGK.HA8 | expression of a V5-tagged anti-CD19 |
| | | CAR. An internal PGK promoter drives |
| | | the expression of an HA8 marker (HA |
| | | epitope presented on a human CD8a |
| | | stalk). |

### Results

Transduced PBMCs were analysed for expression of the transduction markers (RQR8 and HA8) and the V5-tagged CAR. Staining of the PBMCs with antibodies to V5 demonstrated that there was a significant increase in expression when PBMCs were co-transduced with the active VP16- LexA chimeric transcription factor and LexA UAS promoter construct (FIG. 10).

### Example 4

### Design and Testing of a Transcriptional Circuit Using a QF System

The QF system is derived from qa cluster transcription factors controlling the metabolism of quinic acid in the fungus Neurospora crassa. The transcription factor QF positively regulates the expression of genes involved in metabolising quinic acid and the functional domains of the transcription factor have been defined. QF comprises an N-terminal DNA-binding domain, a central domain, proposed to be involved in transcription factor dimerization, and a C-terminal transactivation domain. As the DNA-binding domains and transactivation domains of QF are defined, it is possible to generate a more compact artificial transcription factor, consisting of DBD and TAD fused together, which is considerably smaller than the endogenous transcription factor. This artificial transcription factor is referred to as QF2.

### Constructs

The QF2 system is similar to the GAL4 and LexA systems and comprises a retroviral construct expressing the artificial transcription factor and the sort selection marker RQR8 and a SIN retroviral construct with QF2 UAS promoter (QUAS) driving the expression of a V5-tagged anti-CD19 CAR and a PGK promoter controlling the expression of a HA8 marker (Table 3 and FIG. 11).

TABLE 3
QF2 constructs

| Number | Plasmid name | Description |
|---|---|---|
| AU52483 | SFGmR.RQR8-2A-QF2 | Retroviral construct expressing RQR8 |
| | | sort selection marker and active QF2 |
| | | transcription factor |
| AU52484 | SFGmR.RQR8-2A-QF2_dTAD | Control retroviral construct expressing |
| | | RQR8 sort selection marker and inactive |
| | | QF2 transcription factor consisting of |
| | | DBD alone |
| AU52485 | pSERS11.QUAS.V5- | Self-inactivating (SIN) retroviral vector |
| | aCD19_CAT19_CD8STK- | containing QUAS driving the expression |
| | 41BBz.PGK.HA8-2A-M2 | of a V5-tagged anti-CD19 CAR. An |
| | | internal PGK promoter drives the |
| | | expression of an HA8 marker (HA |
| | | epitope presented on a human CD8a |
| | | stalk). |

Flow cytometric analysis of stained PBMCs with antibodies to HA and RQR8 was carried out to identify transduced populations. Co-transduction of the active QF2 transcription factor with the QUAS promoter construct resulted in the robust expression of the V5-tagged anti-CD19 CAR that was 8-fold higher than controls (FIG. 13). In contrast, V5-tagged anti-CD19 CAR expression was low in PBMCs transduced with QUAS promoter construct alone or in combination with the inactive form of the QF2 transcription factor (QF2-dTAD). Together these results indicate that the QF2 transcription factor is functional in T cells and is capable of driving the expression of a transgene encoding a CAR.

### Example 5

### Design and Testing of a Circular Transcriptional-Dependent Circuit

Circular transcriptional circuits can be generated using a pair of SIN retroviral constructs exhibiting leaky expression from the truncated LTR. Each SIN retroviral construct possesses a UAS with binding sites for an artificial transcription factor, such as GAL4 or LexA, which drives the expression of a marker and/or CAR and an artificial transcription factor. The constructs are designed such that each will have the artificial transcription factor binding to the UAS of the other construct. In this situation, leaky expression from the SIN retroviral construct should lead to the expression of the artificial transcription factor and if the cell is co-transduced with both constructs this should establish a self-amplifying loop that will drive expression from both constructs.

### Constructs

The constructs for the circular transcriptional dependent circuit were generated by modifying the previously described constructs (Table 4 and FIG. 13).

TABLE 4
Circular transcriptional dependent circuit constructs

| Number | Plasmid name | Description |
|---|---|---|
| AU52320 | pSuperRetroW.LexA.RQR8-2A-GAL4 | SIN retroviral vector containing LexA |
| | | UAS driving the expression of a RQR8 |
| | | and GAL4 DNA-binding domain |
| AU52321 | pSuperRetroW.LexA.RQR8-2A-VP16- | SIN retroviral vector containing LexA |
| | GAL4 | UAS driving the expression of a RQR8 |
| | | and active VP16-GAL4 chimeric |
| | | transcription factor |
| AU52329 | pSERS11.Rheo.V5-aCD19_CAT19- | SIN retroviral vector containing GAL4 |
| | CD8STK-41BBz-E2A-HA8-T2A-LexA | UAS driving the expression of a V5- |
| | | tagged anti-CD19 CAR, HA8 marker |
| | | and LexA DNA-binding domain. |
| AU52330 | pSERS11.Rheo.V5-aCD19_CAT19- | SIN retroviral vector containing GAL4 |
| | CD8STK-41BBz-E2A-HA8-T2A-VP16-LexA | UAS driving the expression of a V5- |
| | | tagged anti-CD19 CAR, HA8 marker |
| | | and active VP16-LexA chimeric |
| | | transcription factor. |

### Methods

Retroviral supernatants were generated as described before and used to transduce 293T cells. After 72 hours of transduction the cells were stained with antibodies to the HA8 marker, RQR8 and the V-tagged anti-CD19 CAR.

### Results

Flow cytometric analysis of 293T cells transduced with the individual components of the circular transcriptional dependent circuit showed that expression of the RQR8 and HA8 markers was low, indicating a low level of leaky transcription from the LTRs of the SIN retroviral constructs. When cells were co-transduced with constructs expressing active VP16-GAL4 transcription factor and the GAL4 UAS construct, a higher level of expression of the HA8 marker was observed. To determine if the V5-tagged anti-CD19 CAR was also expressed in the transduced cells, staining with an antibody to the V5 tag and flow cytometric analysis was carried out. This demonstrated that when cells were co-transduced with constructs containing the V5-tagged anti-CD19 CAR under the control of the GAL4 UAS and another construct containing the active VP16-GAL4 transcription factor there was an increase in expression of the CAR with levels 7 to 14-fold higher than those of the single transduction controls.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A kit of vectors for transducing an immune cell with multiple transgenes, the kit comprising:
(i) a first vector which comprises a first transgene and a nucleotide sequence encoding a transcription factor; and
(ii) a second vector which comprises a second transgene
wherein expression of the second transgene within a host cell is dependent upon expression of the transcription factor.

2. A kit of vectors according to claim 1, comprising:
(i) a first vector which comprises a first transgene and a nucleotide sequence encoding a transcription factor;
(ii) a second vector which comprises a second transgene; and
(iii) a third vector which comprises a third transgene
wherein expression of the second and third transgenes within a host cell is dependent upon expression of the transcription factor.

3. A kit of vectors according to claim 1, comprising:
(i) a first vector which comprises a first transgene and a nucleotide sequence encoding a first transcription factor;
(ii) a second vector which comprises a second transgene and a nucleotide sequence encoding a second transcription factor; and
(iii) a third vector which comprises a third transgene
wherein expression of the second transgene within a host cell is dependent upon expression of the first transcription factor, and expression of the third transgene within a host cell is dependent upon expression of the second transcription factor.

4. A kit of vectors according to claim 1, comprising:
(i) a first vector which comprises a first transgene and a nucleotide sequence encoding a first transcription factor; and
(ii) a second vector which comprises a second transgene and a nucleotide sequence encoding a second transcription factor;
wherein expression of the second transgene within a host cell is dependent upon expression of the first transcription factor, and expression of the first transgene within a host cell is dependent upon expression of the second transcription factor.

5. A kit according to claim 1, wherein one or more of the vector(s) also comprises a marker gene.

6. A kit according to claim 1, wherein the vectors are plasmids, transposons, retroviral vectors or lentiviral vectors.

7. A kit according to claim 1 wherein the or each transcription factor is from a non-immune cell or is an artificial transcription factor.

8. A kit according to claim 1, wherein the first vector comprises a constitutively active promoter.

9. A kit according to claim 1, wherein one or more of the transgene(s) encodes a chimeric antigen receptor (CAR) or a T-cell receptor (TCR).

10. A cell which comprises a transgene and a nucleotide sequence encoding a non-endogenous transcription factor, wherein expression of the transgene is dependent upon expression of the non-endogenous transcription factor.

11. A cell transfected or transduced with a kit of vectors according to claim 1.

12. A cell according to claim 11, which is an immune cell.

13. A cell according to claim 12, which is a T-cell or natural killer (NK) cell.

14. A method for making a cell which comprises the step of transducing or transfecting a cell with a kit of vectors according to claim 1.

15. A method for making a cell composition expressing a kit of vectors according to claim 1, which comprises the following steps:
(i) transducing or transfecting a cell with a kit of vectors according to claim 1 wherein one of the vectors comprises a marker gene;
(ii) detecting expression of the marker gene; and
(iii) selecting cells which express the marker gene
wherein expression of the marker gene by a cell indicates that the comprises all of the vectors in the kit

16. A method according to claim 14, wherein the cell(s) is/are transduced or transfected ex vivo.

17. A pharmaceutical composition comprising a plurality of cells according claim 11.

18. A pharmaceutical composition according to claim 17 for use in treating and/or preventing a disease.

19. A method for treating a disease, which comprises the step of administering a pharmaceutical composition according to claim 17 to a subject.

20. A method for treating a disease, which comprises the following steps:
(i) isolation of a cell containing sample from a subject with the disease;
(ii) transduction or transfection of the cells with a kit of vectors according to claim 1; and
(iii) administering the cells from step (ii) to the subject.

21-22. (canceled)
